(19) United States
(12) Patent Application Publication
(10) Pub. No.: US 2022/0184841 A1
(43) Pub. Date: Jun. 16, 2022

(54) PATCH DEVICES, METHODS AND APPARATUS FOR FORMING, AND TESTING PHARMACEUTICAL AGENT DELIVERY PATCH DEVICES

(71) Applicant: BioDelivery Sciences International, Inc., Raleigh, NC (US)

(72) Inventors: Niraj Vasisht, Cary, NC (US); Kenneth E. Schupp, JR., Raleigh, NC (US)

(73) Assignee: BioDelivery Sciences International, Inc., Raleigh, NC (US)

(21) Appl. No.: 17/561,943

(22) Filed: Dec. 25, 2021

Related U.S. Application Data
(63) Continuation of application No. 16/820,655, filed on Mar. 16, 2020.
Application No. 16/820,655 is a continuation of application No. 15/631,393, filed on Jun. 23, 2017, now abandoned.
Application No. 15/631,393 is a continuation of application No. 14/951,647, filed on Nov. 25, 2015, now abandoned.
(60) Provisional application No. 62/092,087, filed on Dec. 15, 2014.
(60) Provisional application No. 62/084,293, filed on Nov. 25, 2014.

(51) Int. Cl. B26F 1/20; A61K 31/485; A61K 9/70; B26F 1/38; B26F 1/14; G01N 33/15; A61K 9/00; A61K 31/4468

(52) U.S. Cl. CPC B26F 1/20; A61K 31/485; A61K 9/7007; B26F 1/384; A61K 31/4468; G01N 33/15; A61K 9/006; A61K 9/7038; B26F 1/14

(57) ABSTRACT

A method for manufacturing a plurality of pharmaceutical agent delivery patch devices includes: providing a supply web including an active layer containing a pharmaceutically active agent; and cutting the supply web to form cut lines through the active layer. The cut lines define in the active layer: a first active layer patch having a first peripheral edge formed by the cutting step; and a second active layer patch having a second peripheral edge formed by the cutting step. The first and second peripheral edges each include a corner portion. At least a first segment of the first peripheral edge and an opposing second segment of the second peripheral edge are formed by a shared one of the cut lines.

### RELATED APPLICATIONS

This application is a continuation of U.S. patent application Ser. No. 15/631,393, filed Jun. 23, 2017, which is a continuation of U.S. patent application Ser. No. 14/951,647, filed Nov. 25, 2015 which claims the benefit of U.S. Provisional Application No. 62/092,087, filed Dec. 15, 2014 and U.S. Provisional Application No. 62/084,293, filed Nov. 25, 2014, the entire contents of each of which are hereby incorporated herein by reference.

### FIELD

The methods described herein relate to pharmaceutical agent delivery patch devices and to methods and apparatus for forming and testing the same.

### BACKGROUND

Various pharmaceutical agent delivery devices, for example, patch devices, are used to administer desired pharmaceutical agents or drugs to a subject (e.g., a human subject). Patches of this type include transdermal delivery patch devices and transmucosal delivery patch devices. Transmucosal patch devices may include patches adapted for oral administration by placement in contact with an oral mucosal surface of a subject.

### SUMMARY

Provided herein are methods for manufacturing a plurality of pharmaceutical agent delivery devices, e.g., patch devices comprising: providing a supply web comprising an active layer comprising a pharmaceutically active agent; and cutting the supply web to form cut lines through the active layer. The cut lines define in the active layer: a first active layer patch having a first peripheral edge formed by the cutting step; and a second active layer patch having a second peripheral edge formed by the cutting step. The first and second peripheral edges each include a corner portion. At least a first segment of the first peripheral edge and an opposing second segment of the second peripheral edge are formed by a shared one of the cut lines.

According to another embodiment, provided herein are methods for manufacturing a plurality of pharmaceutical agent delivery patch devices comprising: conveying a supply web in a conveying direction along a conveying axis, wherein the supply web includes an active layer containing a pharmaceutically active agent; and cutting the supply web to form cut lines through the active layer. The cut lines define in the active layer: a first active layer patch having a first peripheral edge formed by the cutting step; and a second active layer patch having a second peripheral edge formed by the cutting step. At least a first segment of the first peripheral edge and an opposing second segment of the second peripheral edge are formed by a shared one of the cut lines. The first and second active layer patches are disposed in side-by-side relation along a cross-web axis extending transversely to the conveying axis.

According to another embodiment, provided herein are methods for manufacturing a plurality of pharmaceutical agent delivery patch devices includes: conveying a supply web in a conveying direction along a conveying axis, wherein the supply web includes an active layer and a backing layer directly or indirectly bonded to the active layer, the active layer containing a pharmaceutically active agent; and cutting the supply web to form cut lines through the active layer and the backing layer. The cut lines define first and second pharmaceutical agent delivery patch devices in the active layer and the backing layer. The first pharmaceutical agent delivery patch device includes: a first active layer patch having a first peripheral edge formed by the cutting step; and a first backing layer patch directly or indirectly bonded to the first active layer patch. The second pharmaceutical agent delivery patch device includes: a second active layer patch having a second peripheral edge formed by the cutting step; and a second backing layer patch directly or indirectly bonded to the second active layer patch. The method may further include packaging the first and second pharmaceutical agent delivery patch devices in sealed pouches. At least a first segment of the first peripheral edge and an opposing second segment of the second peripheral edge are formed by a shared one of the cut lines.

According to another embodiment, provided herein are methods for manufacturing a plurality of pharmaceutical agent delivery patch devices includes: conveying a supply web in a conveying direction along a conveying axis, wherein the supply web includes an active layer containing a pharmaceutically active agent; and cutting the supply web to form cut lines through the active layer. The cut lines define in the active layer: a first active layer patch having a first peripheral edge formed by the cutting step; and a second active layer patch having a second peripheral edge formed by the cutting step. At least a first segment of the first peripheral edge and an opposing second segment of the second peripheral edge are formed by a shared one of the cut lines. At least the first peripheral edge includes a third edge segment extending obliquely to the conveying direction.

According to another embodiment, provided herein are methods for manufacturing a plurality of pharmaceutical agent delivery patch devices includes: conveying a supply web in a conveying direction along a conveying axis, wherein the supply web includes an active layer containing a pharmaceutically active agent; and cutting the supply web to form cut lines through the active layer. The cut lines define in the active layer: a first active layer patch having a first peripheral edge formed by the cutting step; and a second active layer patch having a second peripheral edge formed by the cutting step. At least a first segment of the first peripheral edge and an opposing second segment of the second peripheral edge are formed by a shared one of the cut lines. The cut lines define waste portions in the active layer between the first and second active layer patches. The method further includes separating the first and second active layer patches from the waste portions.

According to another embodiment, provided herein are methods for manufacturing a plurality of pharmaceutical agent delivery patch devices includes: conveying a supply web in a conveying direction along a conveying axis, wherein the supply web includes an active layer containing a pharmaceutically active agent; and cutting the supply web to form cut lines through the active layer. The cut lines define in the active layer: a first active layer patch having a first peripheral edge formed by the cutting step; and a second active layer patch having a second peripheral edge formed by the cutting step. At least a first segment of the first peripheral edge and an opposing second segment of the second peripheral edge are formed by a shared one of the cut lines. The method further includes thereafter: picking the first and second active layer patches off of the supply web; placing the first and second active layer patches on a packaging apparatus; and packaging the first and second active layer patches in sealed pouches on the packaging apparatus.

Disclosed herein are pharmaceutical agent delivery patches comprising one or more corner edge segments, an effective amount of an opioid drug for the management of pain and/or opioid dependence, wherein the opioid drug is delivered to a mucosal surface in a unidirectional manner, wherein the corner edge segments comprises one or more of a rounded corner edge segment, a circular corner edge segment or a diamond corner edge segment, and wherein when dissolution testing is preformed according to methods presented herein.

Disclosed herein are pharmaceutical agent delivery patches comprising one or more corner edge segments, an effective amount of an opioid drug for the management of pain and/or opioid dependence that dissolves by the process comprising, positioning the pharmaceutical agent delivery patch in a dissolution basket comprising a porous sidewall and a bottom wall defining a basket cavity and a patch holder, such that the patch is supported by the patch device holder and at least a portion of the patch is spaced apart from the sidewall and the bottom wall; immersing the dissolution basket and the patch therein in a dissolution fluid; and moving the dissolution basket with the patch device therein in the dissolution fluid to generate a flow of the dissolution fluid across the patch device, wherein the pharmaceutical agent delivery patch comprising buprenorphine and naloxone, the buprenorphine dissolves between about 54% to about 62% in 10 minutes.

In one embodiment, the buprenorphine dissolves between about 95% and about 100% in about 60 minutes.

In one embodiment, the buprenorphine dissolves between about 83% to about 89% in about 20 minutes.

In one embodiment, the buprenorphine dissolves between about 91% to about 98% in about 30 minutes.

In one embodiment, the buprenorphine dissolves between about 94% to about 100% in 40 minutes.

In one embodiment, the buprenorphine dissolves between about 95% to about 100% in about 50 minutes.

Provided herein are pharmaceutical agent delivery patches comprising one or more corner edge segments, an effective amount of an opioid drug for the management of pain and/or opioid dependence that dissolves by the process comprising, positioning the pharmaceutical agent delivery patch in a dissolution basket comprising a porous sidewall and a bottom wall defining a basket cavity and a patch holder, such that the patch is supported by the patch device holder and at least a portion of the patch is spaced apart from the sidewall and the bottom wall; immersing the dissolution basket and the patch therein in a dissolution fluid; and moving the dissolution basket with the patch device therein in the dissolution fluid to generate a flow of the dissolution fluid across the patch device, wherein the pharmaceutical agent delivery patch comprising buprenorphine alone, the buprenorphine dissolves between about 54% to about 62% in 10 minutes.

In one embodiment, a pharmaceutical agent delivery patch comprising buprenorphine alone dissolves between about 54 to about 62% in 10 minutes and between about 95% to about 100% in about 60 minutes.

In one embodiment, the buprenorphine dissolves between about 10% to about 20% slower in the first 10 to about 30 minutes.

In one embodiment, the buprenorphine dissolves between about 48.6% to about 55.8% in about 10 minutes.

In one embodiment, the buprenorphine dissolves between about 74.7% to about 80.1% in about 20 minutes.

In one embodiment, the buprenorphine dissolves between about 81.9% to about 88.2% in about 30 minutes.

In one embodiment, the buprenorphine dissolves between about 84.6% to about 90% in about 40 minutes.

In one embodiment, the buprenorphine dissolves between about 5.5% to about 100% in about 50 minutes.

Further features, advantages and details of the methods will be provided infra.

### BRIEF DESCRIPTION OF THE DRAWINGS

FIG. 1 is a flow chart of representative embodiments.

FIG. 2 is a top perspective view of a pharmaceutical agent delivery patch device formed using methods described herein.

FIG. 3 is a top view of the pharmaceutical agent delivery patch device of FIG. 2.

FIG. 4 is a schematic diagram of a manufacturing apparatus described herein.

FIG. 5 is a longitudinal cross-sectional view of a supply web for forming the pharmaceutical agent delivery patch device of FIG. 2 using the apparatus of FIG. 4.

FIG. 6 is a bottom view of a cutting die roller forming a part of the apparatus of FIG. 4.

FIG. 7 is a fragmentary, top view of a cut supply web formed by the apparatus of FIG. 4.

FIG. 8 is a longitudinal cross-sectional view of the cut supply web of FIG. 7 taken along the line 8-8 of FIG. 7.

FIG. 9 is an enlarged, fragmentary, top perspective view of the cut supply web of FIG. 7.

FIG. 10 is a schematic diagram of a packaging apparatus for forming packaged pharmaceutical agent delivery patch devices.

FIG. 11 is a top view of a base web of the packaging apparatus of FIG. 10 with pharmaceutical agent delivery patch devices placed thereon.

FIG. 12 is a top perspective view of a packaged pharmaceutical agent delivery patch device.

FIG. 13 is an example of webs with diamond corner edge segments and circular corner edge segments.

FIG. 14 is fragmentary, cross-sectional view of a dissolution testing system according to embodiments presented herein.

FIG. 15 is an exploded, perspective view of a basket assembly forming a part of the dissolution testing system of FIG. 14.

FIG. 16 is a cross-sectional view of the basket assembly of FIG. 15 taken along the line 16-16 of FIG. 15.

FIG. 17 is a top plan view of the basket assembly of FIG. 15.

FIG. 18 is a bottom plan view of the basket assembly of FIG. 15.

FIG. 19 is a front elevational view of a support member forming a part of the basket assembly of FIG. 15.

FIG. 20 is a side elevational view the support member of FIG. 19.

FIG. 21 is a top plan view the support member of FIG. 19.

FIG. 22 is a top perspective view of a pharmaceutical agent delivery patch device to be tested using methods and apparatus according to embodiments presented herein.

FIG. 23 is a top plan view of the pharmaceutical agent delivery patch device of FIG. 22.

FIG. 24 is a top perspective view of a packaged pharmaceutical agent delivery patch device including the pharmaceutical agent delivery patch device of FIG. 22.

FIG. 25 is a top plan view of a basket assembly according to further embodiments presented herein.

FIG. 26 is fragmentary, cross-sectional view of a dissolution testing system according to further embodiments presented herein.

### DETAILED DESCRIPTION OF EMBODIMENTS OF THE INVENTION

The methods presented herein will be described more fully with reference to the accompanying drawings, in which illustrative embodiments are shown. In the drawings, the relative sizes of regions or features may be exaggerated for clarity. The methods described herein, however, may be embodied in many different forms and should not be construed as limited to the embodiments set forth herein; rather, these embodiments are provided so that this disclosure will be thorough and complete, and will fully convey the scope to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings herein.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

As used herein, “monolithic” means an object that is a single, unitary piece formed or composed of a material without joints or seams.

As used herein, “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder (e.g., to alleviate pain).

The term “subject” refers to living organisms such as humans, dogs, cats, and other mammals. Administration of the medicaments included in the devices described herein, which are made by the methods described herein can be carried out at dosages and for periods of time effective for treatment of a subject. In some embodiments, the subject is a human.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, the term “antagonist” refers to a moiety that renders the active agent unavailable to produce a pharmacological effect, inhibits the function of an agonist, e.g., an abusable drug, at a specific receptor, or produces an adverse pharmacological effect. Without wishing to be bound by any particular theory, it is believed that antagonists generally do not alter the chemical structure of the abusable drug itself, but rather work, at least in part, by an effect on the subject, e.g., by binding to receptors and hindering the effect of the agonist. Antagonists can compete with an agonist for a specific binding site (competitive antagonists) and/or can bind to a different binding site from the agonist, hindering the effect of the agonist via the other binding site (non-competitive antagonists). Non-limiting examples of antagonists include opioid neutralizing antibodies; narcotic antagonists such as naloxone, naltrexone and nalmefene; dysphoric or irritating agents such as scopolamine, ketamine, atropine or mustard oils; or any combinations thereof. In one embodiment, the antagonist is naloxone or naltrexone.

The term “impeded” when used to describe the absorption or the delivery of the opioid antagonist from the abuse-resistant device refers to the absorption and/or delivery of said opioid antagonist that is insufficient to inhibit the effects of the opioid agonist comprised in the same device.

As used herein, “addiction therapy” as related to a subject includes the administration of a drug to a subject with the purpose of reducing the cravings for the addictive substance.

As used herein, the term “abusive” or “abusive manner” refers to uses of the devices beyond transmucosal administration such as by injecting or snorting.

As used herein, the term “disposed” refers to the uniform or non-uniform distribution of an element within another.

The term “transmucosal,” as used herein, refers to any route of administration via a mucosal membrane. Examples include, but are not limited to, buccal, sublingual, nasal, vaginal, and rectal. In one embodiment, the administration is buccal. In one embodiment, the administration is sublingual. As used herein, the term “direct transmucosal” refers to mucosal administration via the oral mucosa, e.g., buccal and/or sublingual.

As used herein, the term “water erodible” or “at least partially water erodible” refers to a substance that exhibits a water erodibility ranging from negligible to completely water erodible. The substance may readily dissolve in water or may only partially dissolve in water with difficulty over a long period of time. Furthermore, the substance may exhibit a differing erodibility in body fluids compared with water because of the more complex nature of body fluids. For example, a substance that is negligibly erodible in water may show an erodibility in body fluids that is slight to moderate. However, in other instances, the erodibility in water and body fluid may be approximately the same.

The thicknesses of layers in the drawings may be exaggerated for the purpose of explanation.

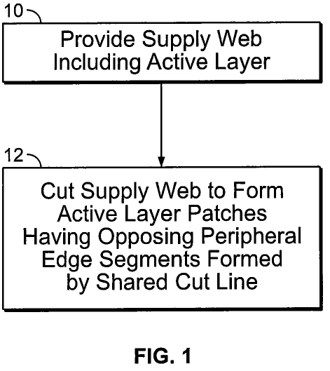

Provided herein are methods or manufacturing processes for manufacturing pharmaceutical agent delivery patches. With reference to FIG. 1, the methods include providing a supply web including an active layer including a pharmaceutically active agent, drug or medicament (Block 10). The supply web is cut to form cut lines through the active layer to define therein at least two active layer patches having opposing peripheral edge segments formed by a shared one of the cut lines (Block 12). In some embodiments, the peripheral edges defining each of the active layer patches each include a curvilinear portion (e.g., a rounded corner). In some embodiments, the two active layer patches are disposed in side-by-side relation across the width of the supply web. In some embodiments, the supply web further includes a backing web that is also cut through to form pharmaceutical agent delivery patches each including one of the active layer patches and a backing layer patch on the active layer patch, and the pharmaceutical agent delivery patch devices are each packaged in sealed pouches. In some embodiments, at least one of the cut lines extends at an oblique angle to the conveying direction. In some embodiments, the cut lines define waste portions in the active layer between the active layer patches, and the active layer patches are removed from the waste portions. In some embodiments, the active layer patches are picked off of the supply web, placed on a packaging apparatus, and packaged in sealed pouches.

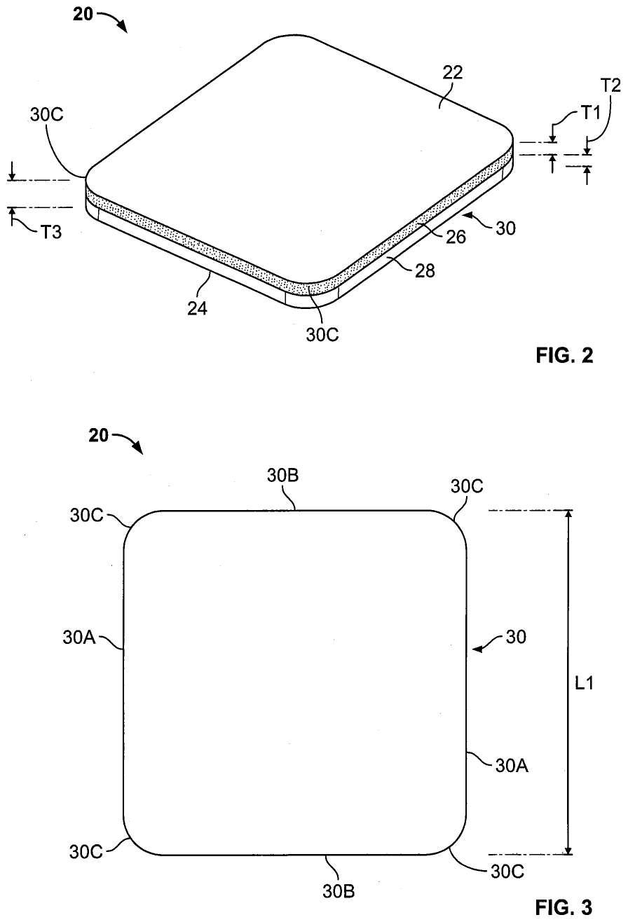

With reference to FIGS. 2 and 3, an exemplary pharmaceutical agent delivery patch device 20 is shown therein and may be formed or manufactured using methods and apparatus according to certain embodiments. The patch device 20 is merely exemplary and, as discussed herein below, may be formed with a different shape, number of layers, dimensions or the like.

The patch device 20 is a laminate structure including a primary or active layer patch 22 superimposed on a secondary or backing layer patch 24. The facing and abutting surfaces of the active layer patch 22 and the backing layer patch 24 are bonded to one another. The layers 22, 24 may be thin layers or films (less than about 500 micrometers), solid and, in some embodiments, flexible. In some embodiments, the layers 22, 24 are self-supporting. The active layer patch 22 contains one or more pharmaceutically active agents, drugs or medicaments. In some embodiments, the active layer patch 22 is a pressure sensitive adhesive (PSA) or mucoadhesive layer. In some embodiments, the patches 22, 24 are monolithic and substantially uniform in thickness and composition across their widths and lengths. In other embodiments, there are two or more active layer patches bonded to one another and bonded to a backing layer patch.

The patch device 20 has and is defined by a circumferential peripheral edge 30, which establishes the shape, boundary or profile of the patch device 20 in two dimensions. The peripheral edge 30 is continuous and endless and includes a pair of opposed longitudinal linear edge segments 30A, a pair of opposed widthwise linear edge segments 30B, and four curvilinear or rounded corner edge segments 30C joining the linear segments 30A, 30B. The patches 22 and 24 are coextensive with the overall patch edge 30 and have corresponding peripheral edges 26 and 28, respectively. The pair of opposed widthwise linear edge segments may be the same length as or a different length than the longitudinal linear edge segments.

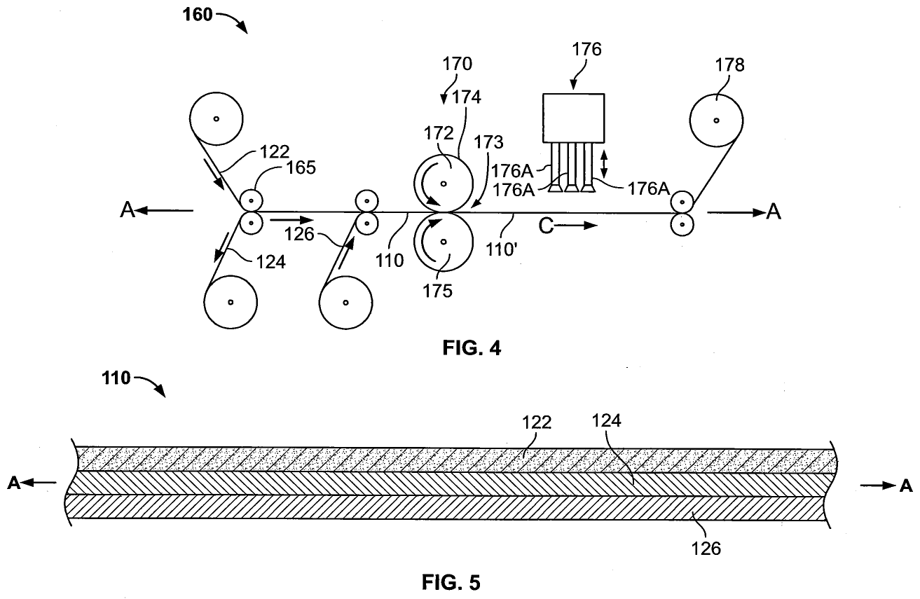

With reference to FIG. 4, a manufacturing line, press or apparatus 160 according to certain embodiments for forming a plurality of the pharmaceutical agent delivery patch devices 20 is shown therein.

See FIG. 13 for alternate examples of patch corner configurations. Examples include circular corner edge segment 1301 configurations and diamond corner edge segment 1302 configurations. The diamond corner configurations, can be, for example, at different angles, for example, from 1 degree to 180 degrees. The circular corner edge segments can any degree of circular between straight and angular.

In accordance with method embodiments herein, a multi-layer, elongate supply web 110 (shown in longitudinal cross-section in FIG. 5) is provided. The supply web 110 includes a flat, elongate primary or active layer web 122 containing one or more pharmaceutically active agents, a flat, elongate secondary or backing layer web 124, and a support web, fabrication liner or release liner 126. The patches 22 and 24 are ultimately formed from the webs 122 and 124, respectively. In some embodiments, the layers 122, 124 are monolithic and substantially uniform in thickness and composition across their widths and lengths.

The supply web 110 may be formed by any suitable method and apparatus, including using any method known in the art. In the illustrative embodiment, the webs 122, 124, 126 are payed out from respective supply rolls. The webs 122 and 124 are mated or laminated by nip rollers 165. The webs 122 and 124 may be bonded together by adhesive properties of one or both of the webs 122, 124, applied heat, applied pressure and/or introduction of an intervening bonding agent or activator (e.g., steam), for example. The combined web 122/124 is then mated or laminated to the release liner 126. The webs 122, 124, 126, 110 are conveyed in a feed, process or conveying direction C along a longitudinal or conveying axis A-A. Various alternatives may be employed. For example, the backing layer web 124 may be laminated to the release liner 126 prior to laminating the active layer web 122 to the web 124.

Other suitable methods for forming the layers 122, 124, 126 as well as the combined web 110 may include, but are not limited to, metered rolling, extrusion, coating, spreading, casting, spraying, and drawing. The layers may be formed in a single-step (e.g., by co-extrusion), and/or by a multi-step coating, spreading, casting, drawing, or a combination thereof. One or more of the layers may be formed (e.g., by coating, spreading, casting, drawing, or a combination thereof) on an already formed layer. Coating or casting methods of forming may include reverse roll coating, gravure coating, immersion or dip coating, metering rod coating, slot die or extrusion coating, gap or knife over roll coating, air knife coating, curtain coating, spin coating, or sputtering. Suitable film forming methods are disclosed in U.S. Pat. No. 8,577,488, the disclosure of which is incorporated herein by reference.

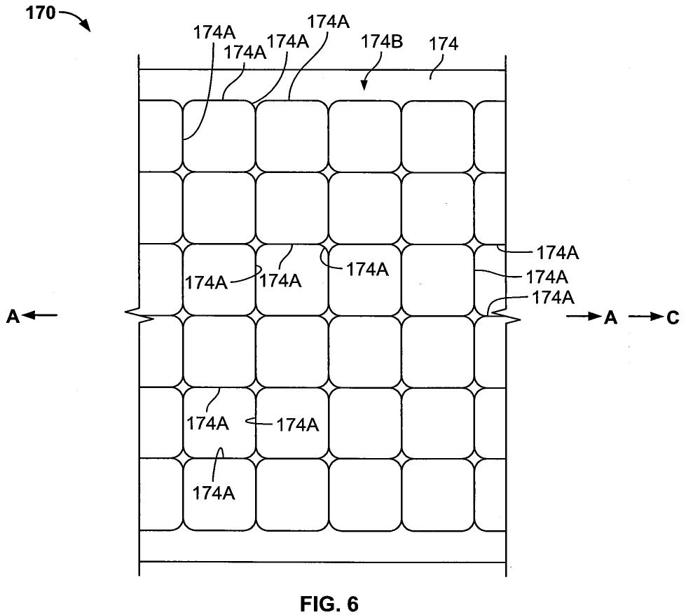

The supply web 110 travels downstream to a cutting station 170. In some embodiments, the cutting station 170 is a rotary die cutter including a rotating die cylinder or roller 172 (having a cutting die surface 174) and a counter-rotating anvil cylinder or roll 175 forming a nip 173 there between. The cutting die surface 174 includes a set 174B of blades 174A (as shown in FIG. 6). The supply web 110 is drawn through the nip 173 in the conveying direction C with the active layer web 122 facing the die roller 172. In this manner, the cutting head 174 continuously, progressively and serially punches, severs or cuts the web 110, forming a cut web 110′.

Other suitable cutting devices may be used in place of the punch cutting apparatus. For example, the cutting station 170 may be a punch cutter including an actuator operable to reciprocatingly press a cutting head (or heads) or die into the supply web 110 against a back plate or platen. The cutting head may similarly include a set of blades corresponding to the blades 174A on the side thereof facing the active layer web 122. The cutting head may be periodically or intermittently pressed into and withdrawn from the web 110 as the web 110 is advanced in the conveying direction C. In this manner, the reciprocating cutting head stepwise and serially punches, severs or cuts the web 110, forming the cut web 110′.

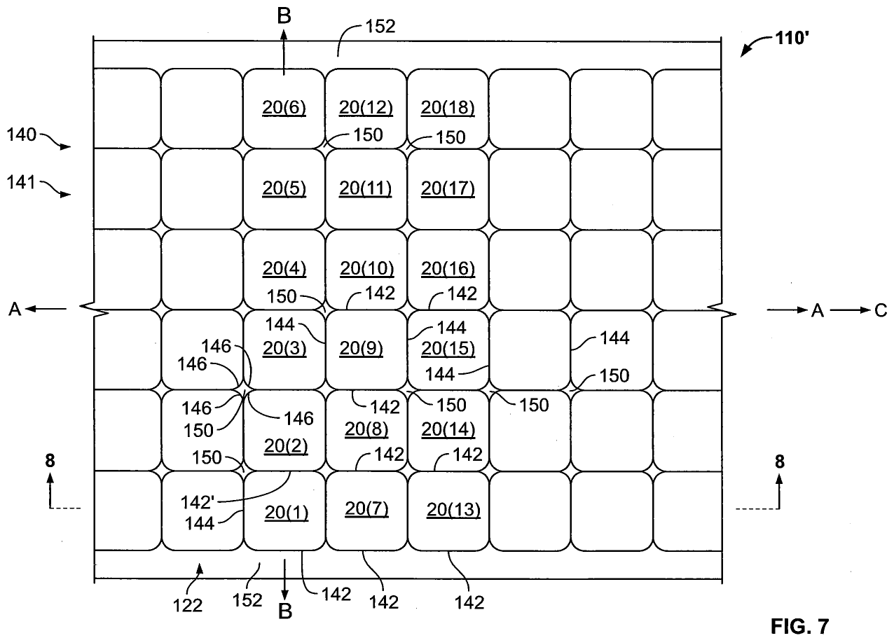

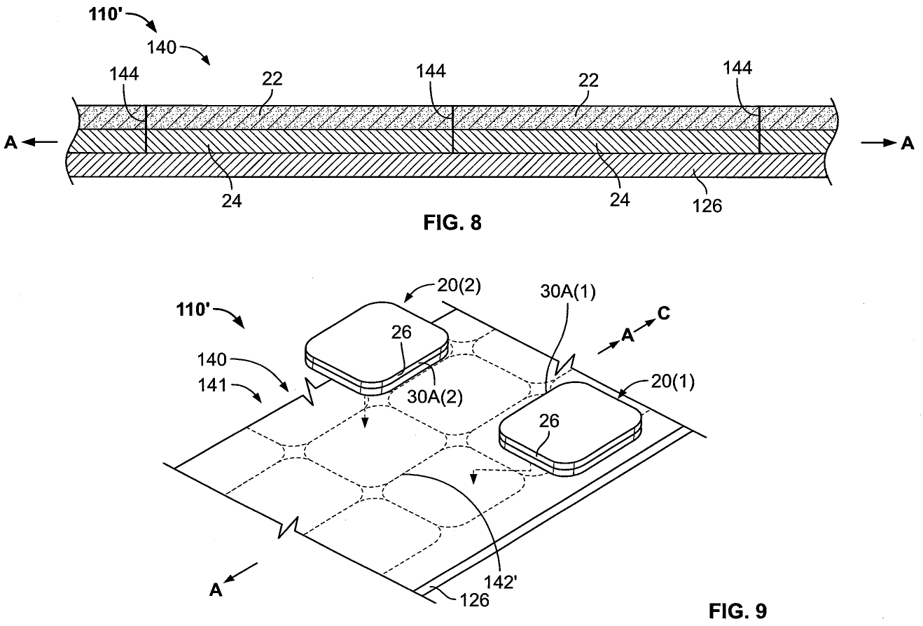

Referring to FIGS. 7 and 8, the cutting blades 174A form a corresponding predetermined set 140 of cut lines 142, 144, 146 in the supply web 110. The cut lines of the set 140 form a cut line pattern 141 and include longitudinal (or conveying or machine direction) cut lines 142, widthwise (or transverse or cross-machine direction) cut lines 144, and corner (or joinder) cut lines 146. In the illustrative exemplary embodiment, the lines 142, 144 are linear and the corner lines 146 are curvilinear, arcuate, radiused or smoothly rounded.

According to some embodiments, the cut lines 142, 144, 146 are “kiss cuts” and extend through the webs 122, 124 but not through the release liner 126, as shown in FIG. 8. In some embodiments, the edges of the blades 174A are sharp so that the cut lines 142, 144, 146 formed thereby are very narrow.

The cut lines 142, 144, 146 form and define in the active layer web 122 and the backing layer web 124 a plurality or array of patch devices 20 (a subset of which are labeled as patch devices 20(1)-20(18) for the purpose of explanation) of predetermined shape, a plurality of interstitial or waste portions 150 between the patch devices, and opposed, longitudinally extending edge border waste portions 152. Thus, the punch cutting die 174 simultaneously forms a plurality or array of the patch devices 20(1)-20(18) and complementary waste portions 150 there between. The array of the patch devices 20(1)-20(18) and complementary waste portions 150 there between may collectively form a tessellation. The illustrated patch devices 20(1)-20(18) have the shape of squares with rounded corner; however, as discussed below, other shapes may be provided. It will be appreciated that the illustrated pattern of patch devices 20 and waste portions 150 may repeat indefinitely.

As can be seen in FIGS. 7 and 8, certain opposing edges of adjacent ones of the patch devices 20(1)-20(18) are formed by a common, same or shared cut line. According to some embodiments, at least some of the patch devices 20(1)-20(18) are formed by multiple cut lines shared with other adjacent ones of the patch devices 20(1)-20(18).

FIG. 9 is an exploded, fragmentary view of the cut web 110′, wherein only the release liner 126 and the patch devices 20(1) and 20(2) are shown for the purpose of explanation. The pattern 141 of the set of cut lines 140 is shown in dashed lines in FIG. 9 for reference. The longitudinal linear edge segment 30A(1) (and the corresponding segment of the active layer patch edge 26) of the patch device 20(1) and the opposing longitudinal linear edge segment 30A(2) (and the corresponding segment of the active layer patch edge 26) of the patch device 20(2) are formed by the same cut line 142′. The remaining linear edge segments 30(A), 30(B) of the patch device 20(2) are likewise formed by the cut lines 142, 144 shared with the patch devices 20 immediately surrounding the patch device 20(2) on its other three sides. The linear edge segments 30A and 30B of the remaining patch devices 20(3)-20(18) are likewise formed by cut lines 142 and 144, respectively, shared with adjacent patch devices with the exception of the linear edge segments 30A proximate the border portions 152.

According to some embodiment, at least some of the cut lines (e.g., the cut lines 146) defining the peripheral edges of the layers 22, 24, 20 extend obliquely to the conveying direction C. That is, at least some of the cut lines generally define an oblique angle with the conveying direction C.

The patch devices 20 formed by shared cut lines may be positioned serially in-line along the conveying direction C and/or may be positioned in side-by-side arrangement extending along a transverse axis B-B (e.g., a widthwise axis transverse (e.g., perpendicular) to the conveying direction C). By way of example, as shown in FIG. 7, the patch devices 20(4)-20(18) are relatively arranged in-line with the conveying direction C and the patch devices 20(1)-20(4) are arranged in cross-web side-by-side relation along the transverse axis B-B. Moreover, the patch device 20(7), for example, is both in-line with the patch devices 20(5), 20(6) and 20(8) and side-by-side with the patch devices 20(3)-20(11).

According to some embodiments, the width of the gap, if any, between the opposing linear edge segments 30A, 30B of adjacent patch devices 20(1)-20(18) is less than 1 mm. In some embodiments, there is no gap or spacing between the opposing linear edge segments 30A, 30B of adjacent patch devices 20(1)-20(18) (i.e., the edge segments 30A, 30B are contiguous or touching one another). In some embodiments, the adjacent, opposing edge segments 30A, 30B are contiguous and remain directly adhered or bonded to one another.

In some embodiments, the edge segments 30A, 30B each have a length L1 (FIG. 3) in the range of from about 0.5 cm to 3 cm.

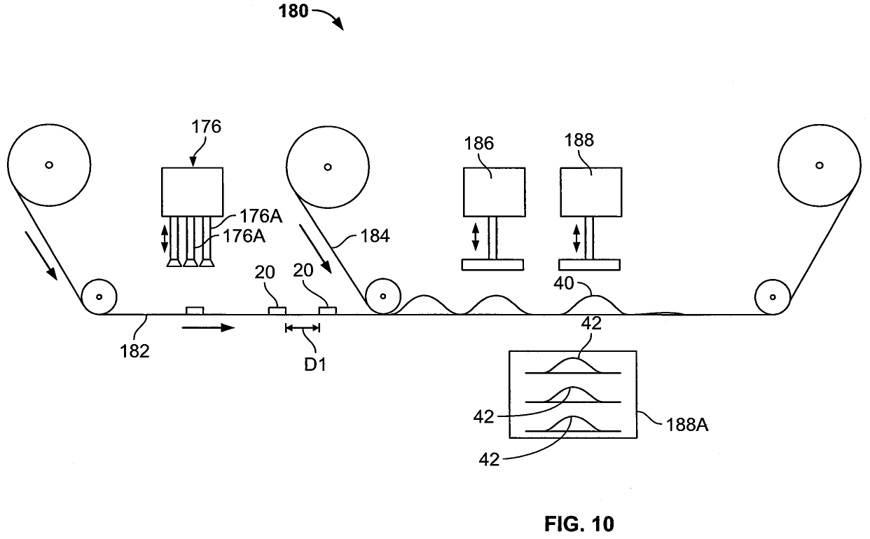

The cut web 110′ is then advanced downstream to a patch removal station 176 configured to remove the cut patch devices 20(1)-20(18) from the release liner 126. In some embodiments, the removal station 176 includes an array of pick up or suction heads or pads 176A arranged and spaced apart in correspondence with the array of patch devices 20(1)-20(18) and operable to pick and place the patch devices. The removal station 176 may include a two-dimensional array (e.g., 4 by 3) of the suction pads 176A. The suction pads 176A engage, vacuum capture and lift (e.g., in batches) the patch devices 20(1)-20(18) off of the release liner 126. The removal station 176 then transports the patches on the suction pads 176A to a base web 182 of a packaging line or apparatus 180 (FIG. 10) and places or deposits the patch devices 20(1)-20(18) on the base web 182 with a predetermined spacing, configuration or distribution.

The remainder of the cut web 110′ may then be taken up on a roll 178 for further processing or disposal. For example, in some embodiments, the waste portions 150, 152 are stripped or otherwise removed from the release liner 126.

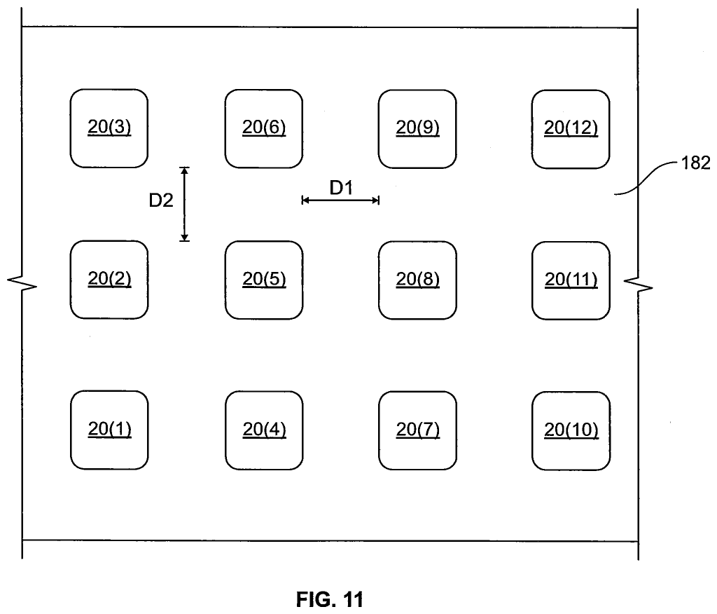

The patch devices 20(1)-20(18) are positioned or deposited on the web 182 in such a manner that each patch device is relatively spaced apart (e.g., longitudinally and laterally) from the adjacent patch devices a predetermined distance D1 as shown in FIG. 11. This may be accomplished in any suitable manner. For example, the suction pads 176A may be moved to deposit the patch devices in spaced relation, the patch devices may be placed on the advancing web 182 at different times to create longitudinal spacing between the placed patch devices, and/or the web 182 may be laterally shifted and the columns of patch devices deposited at different times to create lateral spacing between the placed patch devices.

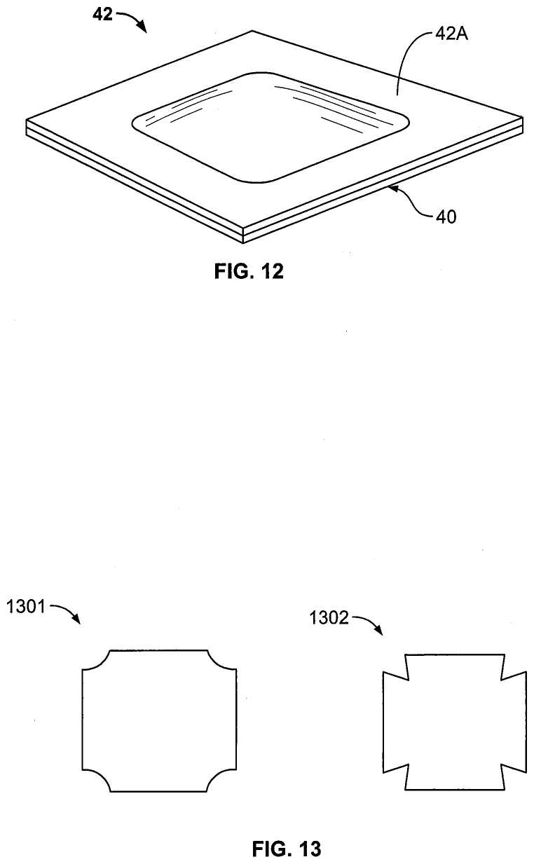

The patch devices 20 on the web 182 are then overlaminated with a second web 184. The webs 182, 184 are sealed about each patch device 20 by a sealing station 186 and cut (e.g., punched) from the web by a cutting station 188 to form a circumferential sealed region 42A about the patch device 20 and thereby a pouch 40 containing the patch device 20. The pouch 40 and the patch device 20 collectively form a packaged pharmaceutical agent delivery patch device 42 (FIG. 12). The packaged devices 42 may be collected in a receptacle 188A. The patch devices 20 may be individually packaged in the pouches.

According to some embodiments, the ratio of {the collective area of the waste portions} per {unit area of the active layer web} generated in forming the patch devices 20 as described herein is less than 30% and, in some embodiments, less than 20%. The waste portions are the portion or portions of the active layer web 122 that do not reside in the active layer patch 22 as defined by the cut lines 140. For example, in the illustrated embodiment, the waste portions consist of the portions 150 and 152.

In some embodiments, the curvilinear edge segments of the patch peripheral edges 30 are convex. In some embodiments, the curvilinear edge segments are rounded corners. In some embodiments, the curvilinear edge segments are arcuate and, in some embodiments, have an arc radius in the range of from about 0.05 mm to 0.5 mm. In some embodiments, the profile of the entirety of each patch peripheral edge 30 is a smooth, continuous curve.

In some embodiments, the active layer patch peripheral edge 26 does not include any sharp corners. In some embodiments, the patch device peripheral edge 30 does not include any sharp corners.

According to some embodiments, each active layer patch 22 has a total area in the range of from about 0.25 cm² to 9 cm². In some embodiments, the total area of each active layer patch 22 does not vary by more than +/−20% between the patch devices 20(1)-20(18) and, in some embodiment, by more than +/−15%, +/−10%, +/−5%, +/−1%, +/−0.5%, +/−0.1%, +/−0.05%, or +/−0.01%.

In some embodiments, each patch 20 has an overall thickness in the range of from about 0.005 to 5 mm and, in some embodiments, in the range of from about 0.05 to 0.5 mm.

According to some embodiments, each active layer patch 22 has a thickness in the range of from about 0.025 to 0.25 mm and, in some embodiments, in the range of from about 0.01 to 0.5 mm.

In some embodiments, each backing layer patch 24 has a thickness in the range of from about 0.1 to 0.3 mm and, in some embodiments, in the range of from about 0.05 to 0.5 mm.

According to some embodiments, the potency per unit area of the pharmaceutical agent contained in the active layer web 122 varies by no more than +/−20% across the width and length of the active layer web 122 and, in some embodiments, by no more than +/−15%, +/−10%, +/−5%, +/−1%, +/−0.5%, +/−0.1%, +/−0.05%, or +/−0.01%. According to some embodiments, the pharmaceutical agent is an opioid (in some embodiments, buprenorphine) and variation in the potency per unit area of the pharmaceutical agent contained in the active layer web 122 is within these ranges.

According to some embodiments, the potency of the pharmaceutical agent in the active layer patches 22 of the patch devices 20(1)-20(18) varies by no more than +/−20% between the patch devices 20(1)-20(18) and, in some embodiments, by no more than +/−15%, +/−10%, +/−5%, +/−1%, +/−0.5%, +/−0.1%, +/−0.05%, or +/−0.01%. According to some embodiments, the pharmaceutical agent is an opioid (in some embodiments, buprenorphine) and variation in the potency of the pharmaceutical agent between the active layer patches 22 is within these ranges.

According to some embodiments, the amount of the pharmaceutical agent in each active layer patch 22 is in the range of from about 50 to 200 μg and, in some embodiments, in the range of from about 10 to 250 μg.

The patch devices 20 may be any suitable type of pharmaceutical agent delivery patch device. Each patch device 20 may be used to treat a subject by administering or delivering an effective dose or amount of the pharmaceutical agent to the subject in a controlled, time release manner.

In some embodiments, the patch devices 20 are transdermal pharmaceutical agent delivery patch device (e.g., a transdermal film) adapted to be adhered to a subject's skin to deliver the pharmaceutical agent to the subject through the skin.

In some embodiments, the patch devices 20 are transmucosal delivery patches adapted to be adhered to a subject to deliver the pharmaceutical agent to the subject via mucosal membrane. According to some embodiments, the patch devices 20 are oral transmucosal delivery patches and, in some embodiments, thin (less than 0.5 mm), flexible, oral transmucosal delivery patches adapted for direct transmucosal administration via buccal or sublingual oral mucosa.

In some embodiments, the patches 20 are sufficiently flexible that they can be bent or rolled.

In some embodiments, the active layer 22 is a layer of a bioerodible mucoadhesive including an effective amount of the pharmaceutically active agent. In some such embodiments, the backing layer 24 is also a bioerodible layer. In some embodiments, the mucoadhesive of the layer 22 is water erodible. In some embodiments, the pharmaceutically active agent contained in the active layer patch 22 is an opioid, and the backing layer 24 includes an opioid antagonist. According to some embodiments, the opioid is buprenorphine and the opioid antagonist is selected from the group consisting of naloxone, naltrexone, nalmefene, nalide, nalmexone, nalorphine, nalbuphine, cyclazocine, levallorphan and combinations thereof.

The patch devices 20 may be or include aspects of devices as disclosed in U.S. Pat. No. 8,147,866 to Finn et al., U.S. Published Patent Applicant No. 2013/0045268 to Finn et al., U.S. Published Patent Applicant No. 2012/0164191 to Finn et al., and U.S. Pat. No. 8,577,488 to Bogue, the disclosures of which are incorporated herein by reference.

While the illustrated patch devices 20 have a shape of a square with rounded corners, any suitable shape may be used. Suitable shapes may include, for example, oval or circular. Other suitable shapes may include a shape that is otherwise a polygon (e.g., a simple, regular or irregular polygon) with any number of sides but has rounded corners or vertices. Suitable polygonal shapes may include, for example, a quadrilateral (including parallelogram, square, rectangular, rhomboid or rhombus), hexagon, octagon, or diamond, or any suitable shape having beveled or truncated corners. In some embodiments, the patch devices 20 are true polygons with any number of sides having non-rounded vertices or sharp vertices.

Thus, it will be appreciated that the waste portions (e.g., the waste portions 150) can have any suitable shape complementary to the patches 30 formed thereabout. Thus, when the corners of the patches are convexly rounded, the sides of the waste portions 150 may be correspondingly concave or scalloped. Waste portions, may be, for example, round, oval, diamond, star or square shaped. There may not be a waste portion, for example, if the patch is a hexagon shaped.

Methods and apparatus according to embodiments described herein can provide a number of advantages while permitting the patches to be formed in a desired shape for ornamental, decorative or functional purposes.

The waste portions of the active layer 122 are reduced. This waste reduction reduces the costs associated with wasted material (including the pharmaceutically active agent), and in some cases, reduces the amount of the pharmaceutical agent not included in the patch devices 20 that must be accounted for and/or addressed. For example, certain pharmaceutical agents such as certain opioids may be tightly regulated and the patch device manufacturer may be required to reconcile, account for and/or specially dispose of waste material including the opioid.

The inventive methods and apparatus can produce patch devices having curvilinear or rounded peripheral edges. In some embodiments, the shape defined by the peripheral edge of each patch device is nonpolygonal. In some embodiments, the peripheral edges of the patch devices are completely devoid of sharp points or transitions. The absence of sharp corners or edges may be desirable ensure comfortable usage and compliance by a subject. Sharp corners may induce significant discomfort when a patch device is applied under the tongue, in the buccal, inside the elbow, vaginally, anally, or the like. Sharp edges may be desirable, see for example, FIG. 13 and description thereof.

For example, in the case of an orally administered (e.g., buccal) patch 20, the relative thickness, flexibility, and smooth edge of the patch 20 can provide relatively minimal mouth feel and little discomfort. This is especially advantageous for patients who have inflammation of the mucosa and/or who may otherwise not be able to comfortably use other devices. The devices are small and flexible enough so that they can adhere to a non-inflamed area of the mucosa and still be effective, e.g., the mucosa does not need to be swabbed with the device.

While the cutting head 174A as described hereinabove forms the cut lines of each patch device 20 in a single cutting or punching step, in other embodiments multiple cutting steps may be used to form some or all of the patch devices 20.

While the supply web 110 as described hereinabove includes a single backing layer 124 and a support web 126, other constructions may be used. For example, in some embodiments additional layers are provided, such as a further, intermediate layer between the active layer 122 and the backing layer 124 or a further layer or layers outside (i.e., above or below) one or both of these layers. In some embodiments, no support web 126 is provided and the active layer 122 (with or without the backing layer 124 or other layers also cut to form parts of the patch device 20) is self-supporting. In some embodiments, the layer patches cut from the supply web (e.g., the patches 22, 24 cut from the supply web 110) form the entirety of the patch device 20 as supplied to the end user as described above. In some embodiments, the active layer patch cut from the supply web is placed on a further layer that also forms a part of the patch device as supplied to the end user. In some embodiments, the active layer patch 22 forms the entirety of the patch device as applied to the end user.

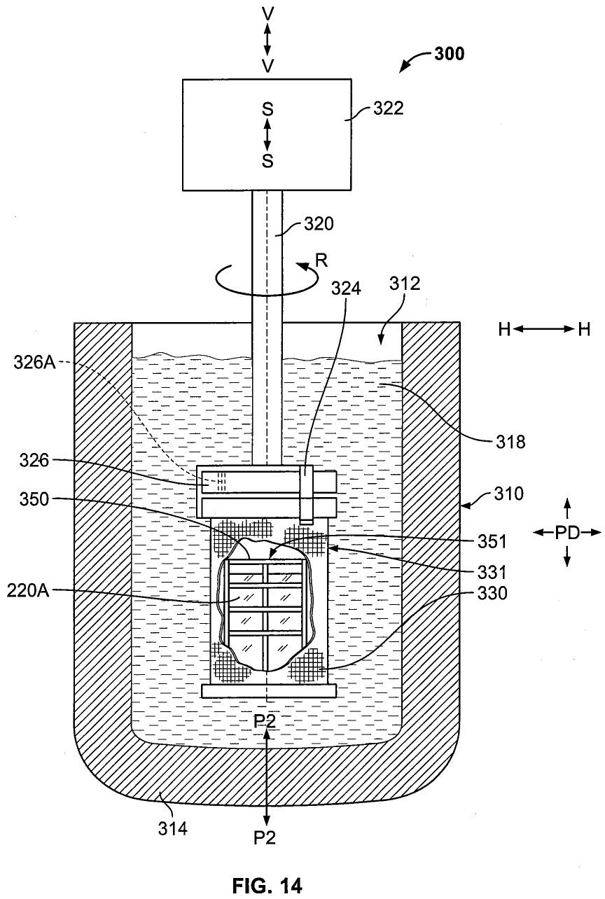

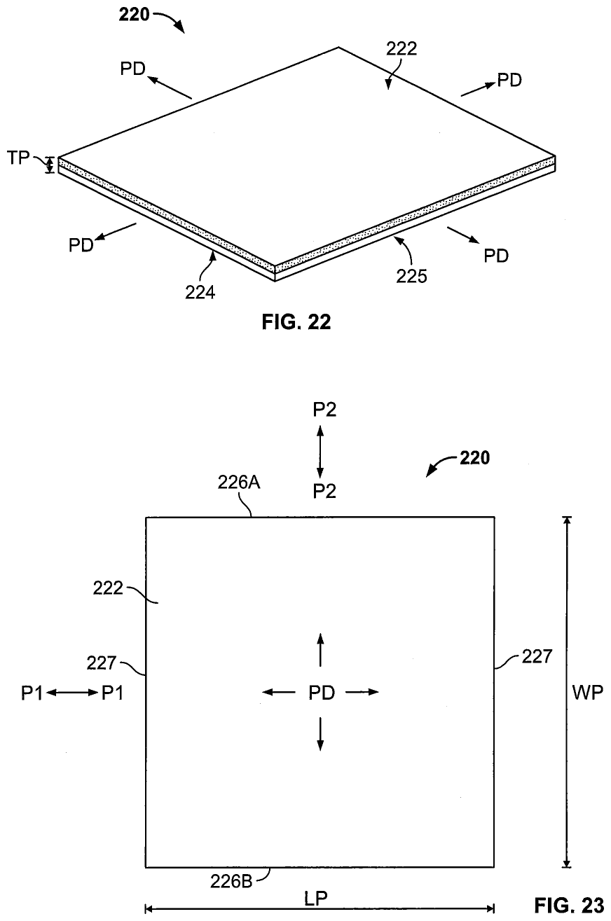

Embodiments provided herein provide methods and apparatus for dissolution testing soluble patch devices such as pharmaceutical agent delivery patches. With reference to FIGS. 14-21, a dissolution testing system 300 according to embodiments provided herein is shown therein. The system 300 and methods described herein may be used to conduct a dissolution test procedure, protocol or monograph on a patch device 220 (FIGS. 22 and 23), for example. In some embodiments, the patch device 220 is a patch device constructed as described above (for example, a patch device 20). As discussed in more detail below, the system 300 includes a patch device holder 350 to support and position the patch device 220 in the system.

With reference to FIGS. 22 and 23, the exemplary patch device 220 is shown therein. The patch device may consist of a single layer with a uniform distribution of active ingredient or multiple layers with differing active ingredients incorporated into or onto the different layers. In some embodiments, the patch device 220 is a pharmaceutical agent delivery patch device. According to some embodiments, the patch device 220 is pliable or flexible at room temperature. The patch device 220 is merely exemplary and, as discussed hereinbelow, may be formed with a different shape, number of layers, dimensions, composition or the like.

The illustrated patch device 220 is a laminate structure including a primary or active layer patch 222 superimposed on a secondary or backing layer patch 224. The facing and abutting surfaces of the active layer patch 222 and the backing layer patch 224 are bonded to one another. The layers 222, 224 may be thin layers or films, solid and pliable or flexible. In some embodiments, the layers 222, 224 are self-supporting. The active layer patch 222 may contain a pharmaceutical active agent, drug or medicament. In some embodiments, the active layer patch 222 is a pressure sensitive adhesive (PSA) or mucoadhesive layer. In some embodiments, the patches 222, 224 are monolithic and substantially uniform in thickness and composition across their widths and lengths.

The overall patch 220 is a relatively thin film or web having a thickness TP that is substantially less than its length LP and width WP. According to some embodiments, the patch length LP and width WP are each at least 2.5 times the patch thickness TP and, in some embodiments at least 300 times the thickness TP. According to some embodiments, the patch device 220 has a thickness TP (FIG. 22) of less than 2 mm, in some embodiments less than 0.05 mm and, in some embodiments in the range of from about 2 mm to 0.05 mm. According to some embodiments, each of the layers 222, 224 has a thickness of less than 2 mm, in some embodiments less than 0.05 mm and, in some embodiments in the range of from about 2 mm to 0.05 mm. In some embodiments, the patch length LP and width WP are each in the range of from about 5 mm to 30 mm.

The patch device 220 has and is defined by a circumferential peripheral edge 225, which establishes the shape, boundary or profile of the patch device 220 in two dimensions. The peripheral edge 225 is continuous and endless and includes a pair of opposed first or longitudinal linear edge segments 226A, 226B, a pair of opposed second or widthwise linear edge segments 227, and four corners joining the edge segments 226A, 226B, 227. The patches 222 and 224 are coextensive with the overall patch edge 225 and have corresponding peripheral edges. The patch device can be of different shapes, for example, square, rectangular, oval, circular, or hexagonal.

The patch device 220 is planar and has a first or longitudinal axis P1-P1 and a perpendicular second or widthwise axis P2-P2 together defining a patch device plane PD-PD (FIGS. 22 and 23).

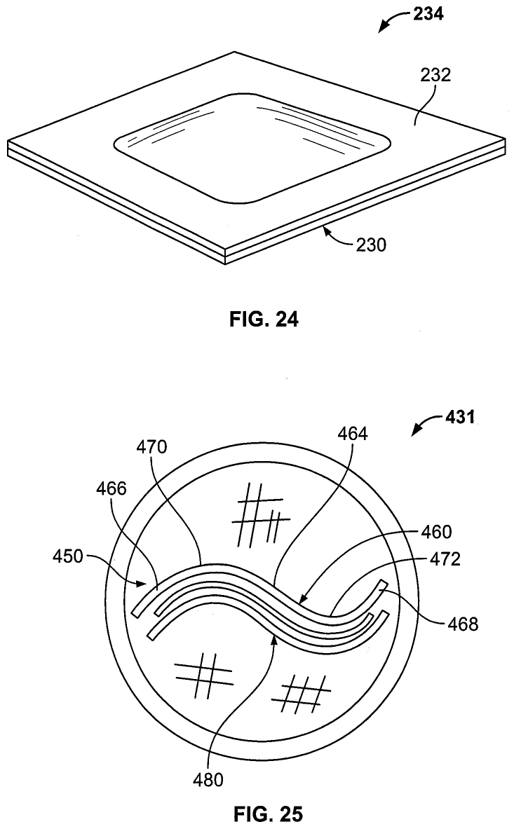

The patch device 220 may be supplied on a release liner from which the patch device can be nondestructively peeled for use. The patch device 220 may be packaged (e.g., individually) in a pouch 230 (FIG. 24) having a circumferential sealed region 232 about the patch device 220. The pouch 230 and the patch device 220 collectively form a packaged pharmaceutical agent delivery patch device 234 (FIG. 24).

The patch device 220 may be manufactured using any suitable method and apparatus. In some methods, thin film webs corresponding to the layers 222, 224 are formed, laminated to one another, or otherwise combined and then cut or punched to form one or more of the patch devices 220. The webs may be formed by metered rolling, extrusion, coating, spreading, casting, spraying, or drawing, for example. The layers may be formed in a single-step (e.g., by co-extrusion), and/or by a multi-step coating, spreading, casting, drawing, or a combination thereof. One or more of the layers may be formed (e.g., by coating, spreading, casting, drawing, or a combination thereof) on an already formed layer. Coating or casting methods of forming may include reverse roll coating, gravure coating, immersion or dip coating, metering rod coating, slot die or extrusion coating, gap or knife over roll coating, air knife coating, curtain coating, spin coating, or sputtering. Suitable film forming methods are disclosed in U.S. Pat. No. 8,577,488, the disclosure of which is incorporated herein by reference.

The layers 222 and 224 may be bonded together by adhesive properties of one or both of the layers 222, 224, applied heat, applied pressure and/or introduction of an intervening bonding agent or activator (e.g., steam), for example.

The dissolution testing system 300 includes a vessel 310, a dissolution medium or fluid 318, a drive shaft 320, a drive mechanism (e.g., an actuator) 322, retention clips 324, a lid 326, a dissolution basket 330, and the patch device holder 350. The vessel 310 includes a bottom wall 314 and defines a vessel cavity 312 containing the dissolution fluid 318. The lid 326 covers the basket 330 and the components 326, 330 are secured to the shaft 320 by the clips 324, and thereby suspended in the vessel cavity 312. The shaft 320 is in turn coupled to the actuator 322, which is operable to forcibly rotate the shaft 320, and thereby the basket 330 about a rotation axis S-S. A vent opening 326A may be provided in the lid 326.

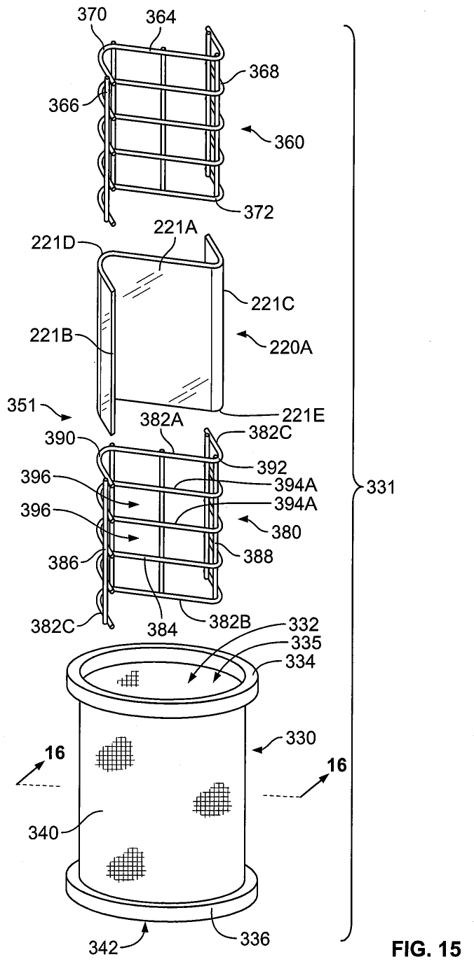

The dissolution basket 330 (FIG. 15) includes an upper ring 334, a lower ring 336, a cylindrical sidewall 340 extending vertically between and secured to each of the rings 334, 336, and a circular bottom wall 342 secured to and covering the opening of the lower ring 336. The side wall 340 and the bottom wall 342 define therein a basket cavity 332 and a top opening 335.

The side wall 340 is porous to permit ample flow of the dissolution fluid 318 therethrough in and out of the basket cavity 332. In some embodiments, the side wall 340 is formed of an open mesh, screen or lattice. The side wall 340 may be formed from crossed wire segments or a perforated sheet, for example. According to some embodiments, the side wall 340 is a screen formed from wires having a wire diameter in the range of from about 0.25 to 0.31 mm and mesh openings having height and width dimensions in the range of from about 0.36 to 0.44 mm. The side wall 340 may be formed of stainless steel, for example.

According to some embodiments, the bottom wall 342 is also porous to permit flow of the dissolution fluid 318 therethrough in and out of the basket cavity 332. The bottom wall 342 may be formed of an open mesh, screen or lattice material having the attributes as described above for the side wall 340.

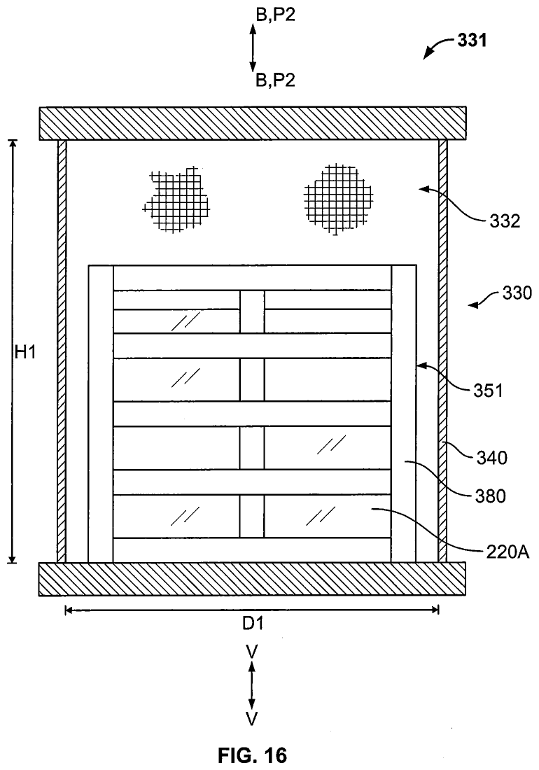

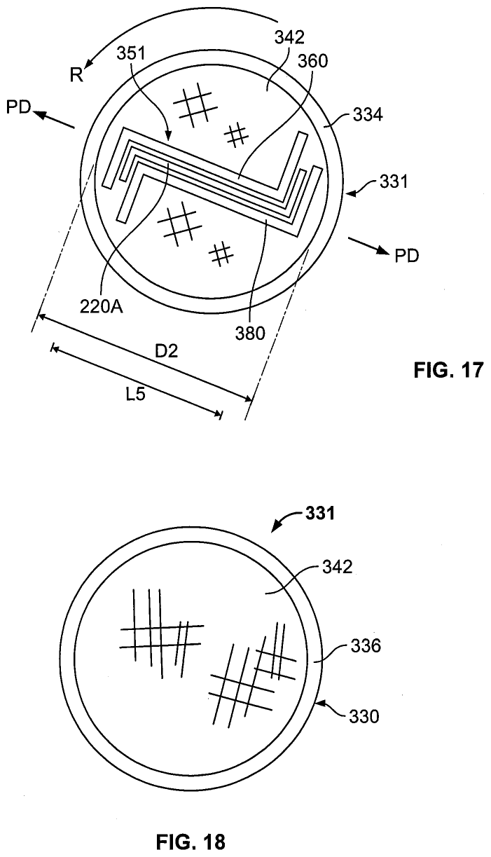

The basket 330 has a vertical basket central axis B-B. According to some embodiments, the inner diameter D2 (FIGS. 16 and 17) of the basket cavity 332 is in the range of from about 19.2 mm to 21.2 mm. According to some embodiments, the height H1 of the basket cavity 332 is in the range of from about 26 mm to 28 mm.

The lid 326 is sized and shaped to close the top opening 335. The lid 326 may be provided with a vent opening 326A. The lid 326 may be formed of any suitable material such as stainless steel.

The basket 330 and the lid 326 (installed on the basket 330) are releasably coupled to the lower end of the shaft 320 by the clips 324.

The actuator 322 may be any suitable device operable to forcibly rotate the shaft 320 about the axis S-S. In some embodiments, the actuator 322 is an electric motor.

The patch device holder 350 includes a first support member 360 and a second support member 380, which cooperate to support and maintain the patch device 220 in a prescribed configuration and orientation.

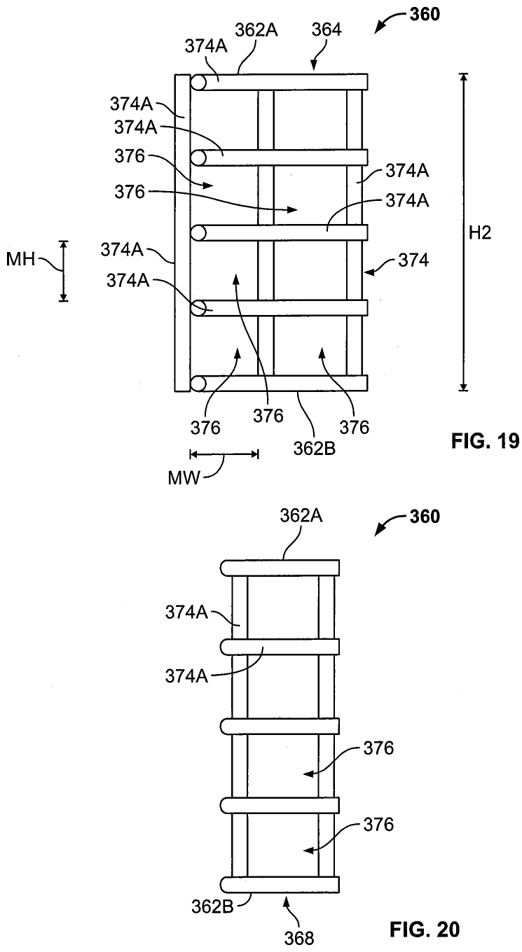

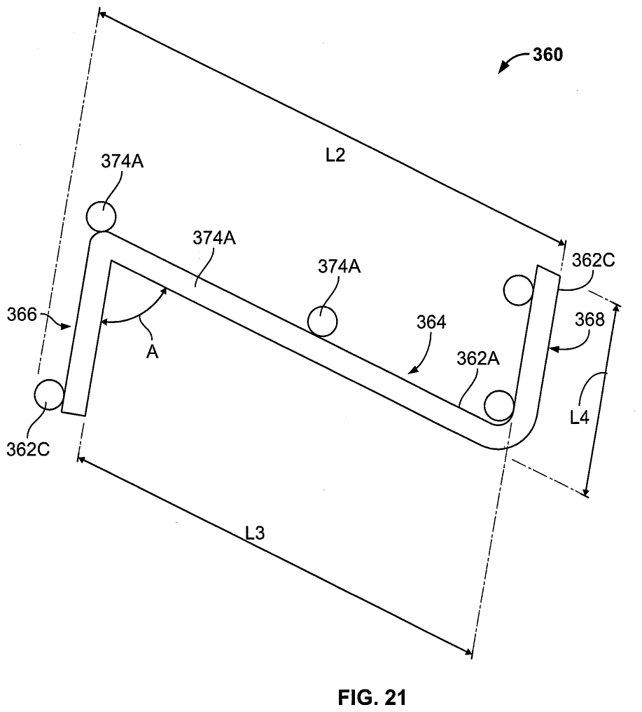

With reference to FIGS. 15 and 19, the first support member 360 has a top edge 362A, a bottom edge 362B, and opposed side edges 362C. The first support member 360 includes a planar center panel 364, an end panel 366 and an end panel 368 joined and delineated by vertical folds 370 and 372. The folds 370, 372 are bent in opposing directions so that the panels 364, 366, 368 collectively form a Z-shaped configuration or profile along a first, horizontal axis H-H. In some embodiments, each end panel 366, 368 forms an included angle A (FIG. 21) with the center panel 364 in the range of from about 10 to 170 degrees, in some embodiments in the range of from about 90 to 135 degrees, in some embodiments in the range of 90 degrees or less, in some embodiments in the range of from about 45 to 90 degrees, and in some embodiments in the range of from 10 to 45 degrees.

The first support member 360 is rigid or semi-rigid and porous to permit flow of the dissolution fluid 318 therethrough. In some embodiments, the first support member 360 is formed of an open mesh, screen or lattice. According to some embodiments and as illustrated, the first support member 360 is formed from crossed vertical and horizontal wire segments 374A (which may be welded, adhered, fused or otherwise bonded at their points of intersection) collectively forming a lattice or screen 374 defining mesh openings 376. Alternatively, the first support member 360 may be formed from a perforated sheet, for example.

According to some embodiments, the wire segments 374A, have a wire diameter of less than 2 mm and, in some embodiments, in the range of from about 0.5 mm to 1.5 mm. According to some embodiments, the mesh openings 376 have height dimensions MH and width dimensions MW in the range of from about 1 mm to 10 mm. According to some embodiments, the area of each mesh opening 376 is in the range of from about 1 to 100 mm2. According to some embodiments, the collective area of the mesh openings 376 constitutes at least 50 percent of the total area of the support member 360 (i.e., the total area of the three panels 364, 366, 368) and, in some embodiments, in the range of from about 10 to 90 percent.

The second support member 380 (FIG. 15) has a top edge 382A, a bottom edge 382B, opposed side edges 382C, a planar center panel 384, an end panel 386, an end panel 388, vertical folds 390, 392, and wire segments 394A defining mesh openings 396, corresponding to the components 362A, 362B, 362C, 364, 366, 368, 370, 372, 374A, and 376, respectively. The folds 390, 392 are bent in opposing directions so that the panels 384, 386, 388 collectively form a Z-shaped configuration or profile. In some embodiments, the second support member 380 is substantially identical to the first support member 360.

The support members 360, 380 may be formed of any suitable material. In some embodiments, the support members 360, 380 are formed of a metal such as stainless steel.

According to some embodiments, the length L2 (FIG. 21) of each support member 360, 380 is in the range of from about 15 mm to 26 mm. According to some embodiments, the height H2 (FIG. 19) of each support member 360, 380 is in the range of from about 15 mm to 26 mm. According to some embodiments, the length L3 (FIG. 21) of the center panel 364 is in the range of from about 2 to 24 mm, and the lengths L4 (FIG. 21) of the end panels 366, 368 are in the range of from about 12 to 2 mm.

The system 300 may be used as follows in accordance with the methods provided herein.

The dissolution fluid 318 is placed in the vessel cavity 312. This step may be executed before or after introducing the basket into the vessel cavity 312.

The patch device 220 to be tested is placed and captured between the support members 360, 380, which are nested together in complementary orientations. That is, the fold 370 is received in the fold 390, the fold 372 is received in the fold 392, the facing panels 364 and 384 are substantially parallel, the facing panels 366 and 386 are substantially parallel, and the facing panels 368 and 388 are substantially parallel, with the patch device 220 sandwiched or interposed therebetween in a space 352. In doing so, the pliable or flexible patch device 220 is bent into a reshaped patch device 220A (FIG. 15) including a center panel 221A and end panels 221B, 221C joined to the center panel 221A at vertical folds 221D, 221E. The center panel 221A is captured between and substantially parallel with the panels 364, 374, the end panel 221B is captured between and substantially parallel with the panels 366, 386, the end panel 221C is captured between and substantially parallel with the panels 368, 388, the fold 221D is captured between the folds 370, 390, and the fold 221E is captured between the folds 372, 392. Accordingly, the patch device 220A assumes the profile of the support members 360, 380. That is, the patch device 220A assumes a fixed, nonplanar configuration and, more particularly, a multi-planar configuration.

The patch device holder 350 (i.e., the support members 360, 380) and the patch device 220A collectively form a supported patch device assembly 351.

The supported patch device assembly 351 is placed in the basket cavity 330 such that the bottom edges 362B, 382B, 226B rest on the bottom wall 342. The assembly 351 remains upright with respect to the basket 330 (axis B-B) and the axis of rotation S-S (which may be aligned with vertical V-V). According to some embodiments and as illustrated, the patch device holder 350 and the basket 330 are relatively sized and shaped such that the assembly 351 is prevented by interference with the basket 330 from falling over beyond a prescribed incline (in some embodiments, the prescribed incline is 10 degrees or less).

According to some embodiments and as shown, the patch device 220A is thereby oriented and maintained upright such that its widthwise axis P2-P2 is substantially parallel to the rotation axis S-S. In some embodiments and as illustrated, the rotation axis S-S lies in the patch device plane PD-PD. In some embodiments, the axis P2-P2 is coaxial with the rotation axis P2-P2 and the rotation axis S-S bisects the patch device 220A into two substantially equal halves. In other embodiments, the patch device 220A may be tilted with respect to the rotation axis S-S such that the patch device axis P2-P2 forms an oblique angle with the rotation axis S-S and, in some embodiments, such that the axis P2-P2 forms an angle with the rotation axis S-S in the range of from about zero to 45 degrees.

The assembly 351 and the basket 330 collectively form a basket assembly 331. The basket assembly 331 is mounted on the end of the shaft 320 using the clips 324 and immersed in the dissolution fluid 318 as shown in FIG. 14.

The actuator 322 rotates the basket assembly 331, and thereby the patch device 220A in a rotation direction R about the rotation axis S-S. According to some embodiments, the rotation axis S-S coincides with the basket center axis B-B. As the basket assembly 331 rotates, a flow of the dissolution fluid 318 is generated across the exposed surfaces of the patch device 220A.

Over time, the dissolution fluid 318, aided by the dissolution fluid flow, causes the patch device 220A to dissolve into the fluid 318. The patch device 220A may be rotated in the fluid 318 for a desired (in some embodiments, prescribed) duration, at the end of which the fluid 318 is tested to determine the concentration of a component dissolved from the patch device 220A in the fluid 318. For example, an aliquot of the fluid 318 may be extracted from the vessel 310 and tested. The patch device 220A may be further rotated in the fluid 318 for a subsequent second desired (in some embodiments, prescribed) duration, at the end of which the fluid 318 is again tested to determine a second concentration of the component in the fluid 318. This procedure may be repeated multiple times over time to compile a dissolution profile (Time versus Concentration of Dissolved Component) for the patch device 220A.

Alternatively, a second patch device 220 of the same specification as the first patch device 220 may be installed and rotated in the dissolution fluid 318 of a second dissolution testing system 300 for a second desired (in some embodiments, prescribed) duration greater than the first duration, at the end of which the fluid 318 of the second system 300 is tested to determine a second concentration of the component in the fluid 318 of the second system 300. This procedure may be executed on additional patch devices 220 each tested using a respective additional system 300 to compile a dissolution profile (Time versus Concentration of Dissolved Component) for the multiple patch devices 220A.

The method may include testing the fluid(s) 318 for the concentration of one or more and, in some embodiments, two or more different components dissolved from the patch device(s) 220. For example, in some embodiments, the method includes testing for the concentrations of a component dissolved from the patch layer 222 and a component dissolved from the patch layer 224. In some embodiments, the method includes testing for the concentration of a pharmaceutically active component dissolved from the patch layer 222 and for the concentration of a pharmaceutically active component dissolved from the patch layer 224.

The patch device holder 350 can be configured and used as an insert that can be selectively placed in and non-destructively removed from the basket cavity 332. In some embodiments, the patch device holder 350 is re-used to dissolution test a subsequent patch device.

The dissolution testing system 300 and patch device holder 350 can provide a number of advantages. In particular, the patch device holder 350 can eliminate or reduce undesirable effects (e.g., hydrodynamic issues) that may be present if the patch device 220 were simply placed in the basket cavity 332. In that case, the patch device 220 may tend to lay flat against the bottom wall 342, lay pressed against the side wall 340 (i.e., semi-cylindrically), or lay in some other inefficient or inconsistent position.

The patch device holder 350 holds the patch device 220 such a portion of the patch device 220 is spaced apart from both the side wall 340 and the bottom wall 342 of the basket 330. In this way, the patch device holder 350 can reduce or prevent the patch device 220 from covering, blocking and occluding the flow openings of the walls 340, 342, which may otherwise tend to reduce the agitation or flow of the dissolution fluid across the patch device 220, and can decrease variability in measurements. Spacing the patch device 220 off the side wall 340 reduce the tendency of the patch device 220 to lie in plane with the direction of flow.

By holding the patch device 220 upright, the patch device holder 350 can prevent air bubbles from being captured under the patch device 220 as may occur if the patch device 220 were laid flat on the bottom wall 342. Such air bubbles may prevent the dissolution fluid from contacting the patch device 220. Also, holding the patch device 220 upright keeps the plane of the patch device 220 from lying flat and orthogonal to the rotation axis S-S(in which case minimal fluid agitation would be provided).

According to some embodiments, at least a portion of the patch device 220A is maintained in spaced apart relation from the side wall 340 and the bottom wall 342. In some embodiments, at least a majority of the patch device 220A is maintained in spaced apart relation from the side wall 340 and the bottom wall 342, in some embodiments, at least 90% and, in some embodiments, as much as 100%. In some embodiments and as shown in the illustrated embodiment, the patch device holder 350 holds the patch device 220A such that the patch device 220A is entirely spaced apart from the side wall 340 and only the bottom edge 226B of the patch device 220A contacts the bottom wall 342. In some embodiments, the patch device 220A is maintained by the patch device holder 350 entirely spaced apart from the walls 340 and 342.

The patch device holder 350 can help to ensure at least a minimal dissolution fluid flow rate is provided across the patch device 220. The patch device holder 350 may provide faster patch device dissolution.

The patch device holder 350 may provide improved testing accuracy, consistency and reproducibility. In particular, the patch device holder 350 can provide more consistent hydrodynamic efficiency between different sizes of patch devices 220 (i.e., the performance of the system 300 is less dependent on patch device size). That is, the system 300 can reduce variability of the rate of dissolution as a function of the size of the patch device. In this way, the system 300 can provide a more uniform dissolution profile between different sizes of patch devices. This may be particularly advantageous because in some instances for each given patch device formulation, the dose and in vivo dissolution rate should be proportional to the patch device size.

Notably, the patch device holder 350 holds the patch device 220A such that as the patch device 220A is rotated both sides thereof are substantially equally exposed to the fluid flow. This equal distribution of flow provides substantially the same dissolution conditions for both layers 222, 224, for example.

The patch device holder 350 can enable effective placement of a patch device 220 in the basket cavity 332, without permitting the patch device 220 to occlude the side wall 340, even when the length LP (FIG. 23) of the patch device 220 exceeds the diameter D5 (FIG. 17) of the cavity 332. As will be appreciated from FIG. 17, the arc lengths of the support members 360, 380, the patch device holder 350 and the supported patch device assembly 351 are greater than the diameter D2 of the cavity 332. However, the length L5 of the supported patch device assembly 351 is less than the basket diameter D2. Thus, a patch device 220 folded to conform to the support members 360, 380 can have a length LP (i.e., the arc length of the reshaped patch device 220A) greater than the diameter D2 of the cavity 332. As a result, the system 300 can accommodate an expanded range of patch device sizes.

According to some embodiments, the arc length and height of each support member 360, 380 exceed the length LP and width WP, respectively, of the patch device 220 so that the supported patch device 220A is fully contained within the periphery or footprint of the support members 360,380.

With reference to FIG. 25, a basket assembly 431 according to further embodiments is shown therein. The basket assembly 431 may be used in place of the basket assembly 331 in the system 100. The basket assembly 431 includes a patch device holder 450 including a first support member 460 and a second support member 480 corresponding to, constructed and used in the same manner as the support members 360, 380 except that the support members 460, 480 are differently shaped.

The first support member 460 includes a curved center panel 464, a curved end panel 466 and a curved end panel 468 joined and delineated by vertical, curved bends 470 and 472. The bends 470, 472 are bent in opposing directions so that the panels 464, 466, 468 collectively form a smooth curve, S-shaped configuration or profile along a horizontal axis. The second support member 480 is likewise configured.

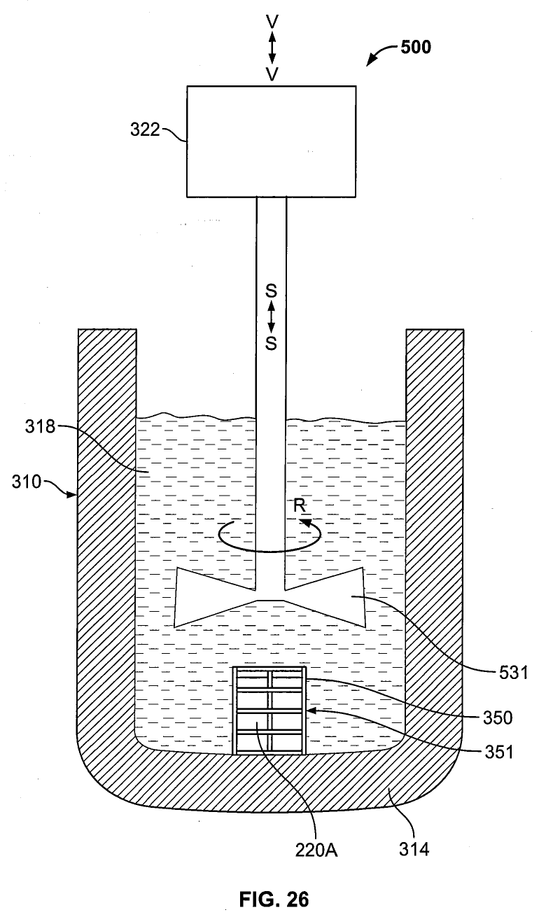

With reference to FIG. 26, a dissolution testing system 500 according to further embodiments is shown therein. The dissolution testing system 500 may be used in place of and in the same manner as the dissolution testing system 500, except as follows.

In the system 500, an agitation paddle 531 is used in place of a dissolution basket. The supported patch device assembly 351 is mounted on the bottom wall 314 of the vessel 310, immersed in the dissolution fluid 318. According to some embodiments and as shown, the patch device holder 350 maintains the patch device 220A in an upright orientation and spaced apart from the bottom wall 314 and side wall of the vessel 310. In some embodiments, the supported patch device assembly 351 is positioned so that it is substantially vertically aligned with the rotation axis S-S.

In use, the actuator 322 rotates the paddle 531 to generate a flow of the fluid 318 across the patch device 220A. The patch device holder 350 positions the patch device 220A in a manner that facilitates efficient and uniform distribution of fluid flow about the patch device 220A and reduces risk of captured air bubbles on the patch device 220A.

While patch device holders 350 and support members have been described herein each having two folds or bends (e.g., folds 370, 372 and bends 470, 472), patch device holders and support members may be provided with more or fewer folds or bends, which may be provided in varying combinations of directions.

The dissolution fluid 318 may include any suitable dissolution fluid. In some embodiments, the dissolution fluid 318 is a liquid. In some embodiments, the dissolution fluid 318 is water. In some embodiments, the dissolution fluid 318 is an aqueous solution. In some embodiments, the dissolution fluid 318 is an acid. In some embodiments, the dissolution fluid is an organic solution (e.g., methanol). Suitable dissolution fluids may include 0.1N Hydrochloric Acid, Simulated Intestinal Fluid, Simulated Gastric Fluid, Simulated Saliva, Phosphate Buffers and Citrate Buffers at various specified pH's.

In some embodiments, each patch 220 has an overall thickness in the range of from about 0.005 to 5 mm and, in some embodiments, in the range of from about 0.05 to 0.5 mm.

According to some embodiments, the amount of the pharmaceutical agent in each active layer patch 222 is in the range of from about 50 μg to 20 mg and, in some embodiments, in the range of from about 10 μg to 25 mg.

The patch devices 220 may be any suitable type of pharmaceutical agent delivery patch device. Each patch device 220 may be used to treat a subject by administering or delivering an effective dose or amount of the pharmaceutical agent to the subject in a controlled, time release manner.

In some embodiments, the patch devices 220 are transdermal pharmaceutical agent delivery patch device (e.g., a transdermal film) adapted to be adhered to a subject's skin to deliver the pharmaceutical agent to the subject through the skin.

In some embodiments, the patch devices 220 are transmucosal delivery patches adapted to be adhered to a subject to deliver the pharmaceutical agent to the subject via mucosal membrane. According to some embodiments, the patch devices 220 are oral transmucosal delivery patches and, in some embodiments, thin (less than 2 mm), flexible, oral transmucosal delivery patches adapted for direct transmucosal administration via buccal or sublingual oral mucosa.

In some embodiments, the patches 220 are sufficiently flexible that they can be bent or rolled.

In some embodiments, the active layer 222 is a layer of a bioerodible mucoadhesive including an effective amount of the pharmaceutical active agent. In some such embodiments, the backing layer 224 is also a bioerodible layer. In some embodiments, the mucoadhesive of the layer 222 is water erodible. In some embodiments, the pharmaceutical active agent contained in the active layer patch 222 is an opioid, and the backing layer 224 includes an opioid antagonist. According to some embodiments, the opioid is buprenorphine and the opioid antagonist is selected from the group consisting of naloxone, naltrexone, nalmefene, nalide, nalmexone, nalorphine, nalbuphine, cyclazocine, levallorphan and combinations thereof.

The patch devices 220 may be or include aspects of devices as disclosed in U.S. Pat. No. 8,147,866 to Finn et al., U.S. Published Patent Applicant No. 2013/0045268 to Finn et al., U.S. Published Patent Applicant No. 2012/0164191 to Finn et al., and U.S. Pat. No. 8,577,488 to Bogue, the disclosures of which are incorporated herein by reference.

While the illustrated patch devices 220 have a shape of a square, any suitable shape may be used. Suitable shapes may include, for example, oval or circular. Other suitable shapes may include a polygon (e.g., a simple, regular or irregular polygon). Suitable polygonal shapes may include, for example, a quadrilateral (including parallelogram, square, rectangular, rhomboid or rhombus), hexagon, octagon, or diamond, or any suitable shape.

The patch 220 may have more or fewer than two layers 222, 224.

In one embodiment, a pharmaceutical agent delivery patch comprising one or more corner edge segments, an effective amount of an opioid drug for the management of pain and/or opioid dependence that dissolves by the process comprising, positioning the pharmaceutical agent delivery patch in a dissolution basket comprising a porous sidewall and a bottom wall defining a basket cavity and a patch holder, such that the patch is supported by the patch device holder and at least a portion of the patch is spaced apart from the sidewall and the bottom wall; immersing the dissolution basket and the patch therein in a dissolution fluid; and moving the dissolution basket with the patch device therein in the dissolution fluid to generate a flow of the dissolution fluid across the patch device, wherein the pharmaceutical agent delivery patch dissolved as shown in Table 1.

In one embodiment, a pharmaceutical agent delivery comprising buprenorphine and naloxone, the buprenorphine dissolves between about 54% to about 62% in 10 minutes and between about 95% and about 100% in about 60 minutes. It may also dissolve between about 83% to about 89% in about 20 minutes. It may also dissolve between about 91% to about 98% in about 30 minutes. It may also dissolve between about 94% to about 100% in 40 minutes. It may also dissolve between about 95% to about 100% in about 50 minutes.

In other embodiments, a pharmaceutical agent delivery comprising buprenorphine alone dissolves between about 54-62% in 10 minutes and between about 95-100% in 60 minutes. In other embodiments, if the patch is larger and the amount of buprenorphine is lower, the buprenorphine will dissolve about 10% slower than the values shown in Table 1 for the first few time points. In other embodiments, if the patch has a lower concentration of buprenorphine, the buprenorphine will dissolve about 5% to about 20% slower, about 5% slower, about 10% slower, or about 10% to about 20% slower than the values shown in Table 1 for the first few time points. For example, a pharmaceutical agent delivery comprising buprenorphine alone with a lower concentration of buprenorphine per dosage form than the products of Table 1 dissolves between about 48.6 to about 55.8% in about 10 minutes, between about 74.7% to about 80.1% in about 20 minutes. It may also dissolve between about 81.9% to about 88.2% in about 30 minutes. It may also dissolve between about 84.6% to about 90% in about 40 minutes. It may also dissolve between about 85.5% to about 100% in about 50 minutes. For example, a pharmaceutical agent delivery comprising buprenorphine alone with a lower concentration of buprenorphine per dosage form than the products of Table 1, dissolves between about 43.2 to about 48.8% in about 10 minutes, between about 66% to about 71.2% in about 20 minutes. It may also dissolve between about 72.8% to about 77.6% in about 30 minutes. It may also dissolve between about 75.2% to about 100% in about 40 minutes. It may also dissolve between about 76% to about 100% in about 50 minutes.

For example, a pharmaceutical agent delivery comprising buprenorphine alone with a lower concentration of buprenorphine per dosage form than the products of Table 1 dissolves between about 52% to about 58.6% in about 10 minutes, between about 79% to about 84% in about 20 minutes. It may also dissolve between about 86% to about 92% in about 30 minutes. It may also dissolve between about 89.3% to about 100% in about 40 minutes. It may also dissolve between about 90% to about 100% in about 50 minutes.

Table 1 below demonstrates the dissolution of the three shown amounts of buprenorphine and naloxone in a combination patch at certain concentrations.

TABLE 1

| | Strength | | |
|---|---|---|---|
| | (buprenorphine/naloxone) | | |
| | 2.1 mg/0.3 mg | 4.2 mg/0.7 mg | 6.3 mg/1.0 mg |
| | n = | | |
| | 6 | 6 | 6 |
| | % Dissolved | % Dissolved | % Dissolved |
| BUP-10 min AVE | 61.7 | 59.0 | 54.8 |
| BUP-20 min AVE | 86.7 | 88.5 | 83.5 |
| BUP-30 min AVE | 92.8 | 97.2 | 91.8 |
| BUP-40 min AVE | 94.7 | 99.3 | 94.3 |
| BUP-50 min AVE | 95.3 | 100.2 | 95.0 |
| BUP-60 min AVE | 95.5 | 100.7 | 95.3 |
| NAL-10 min AVE | 70.5 | 69.7 | 68.7 |
| NAL-20 min AVE | 90.3 | 88.2 | 91.2 |
| NAL-30 min AVE | 95.2 | 94.8 | 97.0 |
| NAL-40 min AVE | 96.8 | 96.3 | 98.2 |
| NAL-50 min AVE | 97.2 | 97.3 | 99.0 |
| NAL-60 min AVE | 97.8 | 97.5 | 99.2 |

Disclosed herein are pharmaceutical agent delivery patches comprising one or more corner edge segments, an effective amount of an opioid drug for the management of pain and/or opioid dependence, wherein the opioid drug is delivered to a mucosal surface in a unidirectional manner, wherein the corner edge segments comprises one or more of a rounded corner edge segment, a circular corner edge segment or a diamond corner edge segment, and wherein when dissolution testing is preformed according to methods presented herein.

Disclosed herein are pharmaceutical agent delivery patches comprising one or more corner edge segments, an effective amount of an opioid drug for the management of pain and/or opioid dependence that dissolves by the process comprising, positioning the pharmaceutical agent delivery patch in a dissolution basket comprising a porous sidewall and a bottom wall defining a basket cavity and a patch holder, such that the patch is supported by the patch device holder and at least a portion of the patch is spaced apart from the sidewall and the bottom wall; immersing the dissolution basket and the patch therein in a dissolution fluid; and moving the dissolution basket with the patch device therein in the dissolution fluid to generate a flow of the dissolution fluid across the patch device, wherein the pharmaceutical agent delivery patch comprising buprenorphine and naloxone, the buprenorphine dissolves between about 54% to about 62% in 10 minutes.

In one embodiment, the buprenorphine dissolves between about 95% and about 100% in about 60 minutes.

In one embodiment, the buprenorphine dissolves between about 83% to about 89% in about 20 minutes.

In one embodiment, the buprenorphine dissolves between about 91% to about 98% in about 30 minutes.

In one embodiment, the buprenorphine dissolves between about 94% to about 100% in 40 minutes.

In one embodiment, the buprenorphine dissolves between about 95% to about 100% in about 50 minutes.

Provided herein are pharmaceutical agent delivery patches comprising one or more corner edge segments, an effective amount of an opioid drug for the management of pain and/or opioid dependence that dissolves by the process comprising, positioning the pharmaceutical agent delivery patch in a dissolution basket comprising a porous sidewall and a bottom wall defining a basket cavity and a patch holder, such that the patch is supported by the patch device holder and at least a portion of the patch is spaced apart from the sidewall and the bottom wall; immersing the dissolution basket and the patch therein in a dissolution fluid; and moving the dissolution basket with the patch device therein in the dissolution fluid to generate a flow of the dissolution fluid across the patch device, wherein the pharmaceutical agent delivery patch comprising buprenorphine alone, the buprenorphine dissolves between about 54% to about 62% in 10 minutes.

In one embodiment, a pharmaceutical agent delivery patch comprising buprenorphine alone dissolves between about 54 to about 62% in 10 minutes and between about 95% to about 100% in about 60 minutes.

In one embodiment, the buprenorphine dissolves between about 10% to about 20% slower in the first 10 to about 30 minutes.

In one embodiment, the buprenorphine dissolves between about 48.6% to about 55.8% in about 10 minutes.

In one embodiment, the buprenorphine dissolves between about 74.7% to about 80.1% in about 20 minutes.

In one embodiment, the buprenorphine dissolves between about 81.9% to about 88.2% in about 30 minutes.

In one embodiment, the buprenorphine dissolves between about 84.6% to about 90% in about 40 minutes.

In one embodiment, the buprenorphine dissolves between about 5.5% to about 100% in about 50 minutes.

The pharmaceutically active agent used in the active layer patch (e.g., the active layer patch 222) may include any suitable pharmaceutically active agent(s). In some embodiments, the pharmaceutically active agent includes an antacid, H2-antagonist, or an analgesic. The pharmaceutically active agent may include one or more of the pharmaceutically active agents listed or described in U.S. Pat. No. 8,577,488 to Bogue, the disclosure of which is incorporated herein by reference.

According to some embodiments, a pharmaceutically active agent or a medicament for use in the active layer patch (e.g., the active layer patch 222) includes any medicament capable of being administered transmucosally. The medicament can be suitable for local delivery to a particular mucosal membrane or region, such as the buccal and nasal cavities, throat, vagina, alimentary canal or the peritoneum. Alternatively, the medicament can be suitable for systemic delivery via such mucosal membranes.

In some embodiments, the pharmaceutically active agent medicament is an opioid. Opioids suitable for use include, e.g., alfentanil, allylprodine, alphaprodine, apomorphine, anileridine, apocodeine, benzylmorphine, bezitramide, buprenorphine, butorphanol, clonitazene, codeine, cyclorphan, cyprenorphine, desomorphine, dextromoramide, dextropropoxyphene, dezocine, diampromide, diamorphone, dihydrocodeine, dihydromorphine, dimenoxadol, eptazocine, ethylmorphine, etonitazene, etorphine, fentanyl, fencamfamine, fenethylline, hydrocodone, hydromorphone, hydroxymethylmorphinan, hydroxypethidine, isomethadone, levomethadone, levophenacylmorphan, levorphanol, lofentanil, mazindol, meperidine, metazocine, methadone, methylmorphine, modafinil, morphine, nalbuphene, necomorphine, normethadone, normorphine, opium, oxycodone, oxymorphone, pholcodine, profadol remifentanil, sufentanil, tramadol, corresponding derivatives, physiologically acceptable compounds, salts and bases. In some embodiments, the medicament is fentanyl, e.g., fentanyl citrate. In some embodiments, the medicament is buprenorphine.

The amount of medicament, e.g., fentanyl or buprenorphine, to be incorporated into the device depends on the desired treatment dosage to be administered, e.g., the fentanyl or fentanyl derivative can be present in about 0.001% to about 50% by weight of the patch device, and in some embodiments between about 0.005 and about 35% by weight or the buprenorphine can be present in about 0.001% to about 50% by weight of the device, and in some embodiments between about 0.005 and about 35% by weight. In one embodiment, the patch device (e.g., the patch device 20) comprises about 3.5% to about 4.5% fentanyl or fentanyl derivative by weight. In one embodiment, the patch device comprises about 3.5% to about 4.5% buprenorphine by weight. In another embodiment, the patch device comprises about 800 μg of a fentanyl such as fentanyl citrate. In another embodiment the device comprises about 25, 50, 75, 100, 150, 200, 300, 400, 500, 600, 700, 900, 1000, 1200, 1500, 1600 or 2000 μg of a fentanyl such as fentanyl citrate or fentanyl derivative. It is to be understood that all values and ranges between these values and ranges are meant to be encompassed. In another embodiment, the patch device comprises about 800 μg of buprenorphine. In another embodiment, the patch device comprises about 100, 200, 300, 400, 500, 600, 700, 900, 1000, 1200, 1500, or 2000 μg of buprenorphine. In another embodiment, the patch device comprises about 25, 50, 75, 100, 150, 200, 300, 400, 500, 600, 700, 900, 1000, 1200, 1500, 1600 or 2000 μg of any of the medicaments described herein.

In certain embodiments and aspects described herein, abuse-resistant transmucosal delivery devices are formed according to the methods disclosed herein. Accordingly, in one embodiment, the patch devices manufactured are abuse-resistant mucoadhesive delivery devices suitable for administration of an effective amount of an opioid drug to a subject, for the management of pain and/or opioid dependence. The patch device is capable of delivering the opioid agonist by means of a unidirectional gradient (i.e., flux that flows toward the mucosa) that is created upon application of the device to a mucosal surface.

The patch devices manufactured in accordance with methods described herein can include any combination or sub-combination of ingredients, layers and/or compositions of, e.g., the devices described in U.S. Pat. Nos. 6,159,498, 5,800,832, 6,585,997, 6,200,604, 6,759,059 and/or PCT Publication No. WO 05/06321. The contents of these patent and publications are incorporated herein by reference in their entireties.

In some embodiments, the active layer patch (e.g., active layer patch 22) mucoadhesive layer that is a bioerodable or water-erodable mucoadhesive layer. In some embodiments, the active layer patch includes a bioerodable mucoadhesive layer which comprises a mucoadhesive polymeric diffusion environment. The active layer patch adheres to a mucosal surface of the subject within about 5 seconds following application.

In some embodiments, mucoadhesive polymeric diffusion environment comprises an opioid agonist. In some embodiments, the opioid agonist is buprenorphine. In some embodiments related to the treatment of opioid dependence, the dose of buprenorphine that can be incorporated into the devices described herien depends on the desired treatment dosage to be administered and can range from about 10 μg to about 20 mg of buprenorphine. In other embodiments related to the treatment of pain, the dose of buprenorphine can range from about 60 μg to about 6 mg. In some embodiments, the low dose of buprenorphine comprised in the mucoadhesive device is between about 0.075 and 12 mg of buprenorphine, e.g., 0.075 mg, 0.080 mg, 0.085 mg, 0.090 mg, 0.095 mg, 0.10 mg, 0.15 mg, 0.20 mg, 0.25 mg, 0.30 mg, 0.35 mg, 0.40 mg, 0.45 mg, 0.50 mg, 0.44 mg, 0.60 mg, 0.65 mg, 0.70 mg, 0.75 mg, 0.80 mg, 0.85 mg, 0.90 mg, 0.95 mg, 1.00 mg, 1.5 mg, 1.75 mg, 2.0 mg, 2.5 mg, 3.0 mg, 3.5 mg, 4.0 mg, 4.5 mg, 5.0 mg, 5.25 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg or 12.0 mg or buprenorphine. In one embodiment, the dose is 0.875 mg of buprenorphine. In another embodiment, the dose is 1.75 mg of buprenorphine. In another embodiment, the dose is 3.5 mg of buprenorphine. In yet another embodiment, the dose is 5.25 mg of buprenorphine.

In some embodiments, the mucoadhesive polymeric diffusion environment can include the drug, at least one pharmacologically acceptable polymer capable of bioadhesion (the “bioadhesive polymer”), and can optionally include at least one film-forming bioerodable or water-erodable polymer (the “film-forming polymer”). Alternatively, the mucoadhesive polymeric diffusion environment can be formed of a single polymer that acts as both the bioadhesive polymer and the first film-forming polymer. Additionally or alternatively, the mucoadhesive polymeric diffusion environment can include other film-forming water-erodable polymers and/or water-erodable plasticizers, such as glycerin and/or polyethylene glycol (PEG).

In some embodiments, the bioadhesive polymer of the mucoadhesive polymeric diffusion environment can include any water erodable substituted cellulosic polymer or substituted olefinic polymer wherein the substituents may be ionic or hydrogen bonding, such as carboxylic acid groups, hydroxyl alkyl groups, amine groups and amide groups. For hydroxyl containing cellulosic polymers, a combination of alkyl and hydroxyalkyl groups will be preferred for provision of the bioadhesive character and the ratio of these two groups will have an effect upon water swellability and dispersability. Examples include polyacrylic acid (PAA), which can optionally be partially cross-linked, sodium carboxymethyl cellulose (NaCMC), moderately to highly substituted hydroxypropylmethyl cellulose (HPMC), polyvinylpyrrolidone (PVP3 which can optionally be partially cross-linked), moderately to highly substituted hydroxyethylmethyl cellulose (HEMC) or combinations thereof. In one embodiment, HEMC can be used as the bioadhesive polymer and the first film forming polymer as described above for a mucoadhesive polymeric diffusion environment formed of one polymer. These bioadhesive polymers are preferred because they have good and instantaneous mucoadhesive properties in a dry, system state.

In some embodiments, the mucoadhesive polymeric diffusion environment, e.g., a bioerodable mucoadhesive layer, is generally comprised of water-erodable polymers which include, but are not limited to, hydroxyethyl cellulose (HEC), hydroxypropyl cellulose (HPC), hydroxypropylmethyl cellulose (HPMC), hydroxyethylmethyl cellulose (HEMC), polyacrylic acid (PAA) which may or may not be partially cross-linked, sodium carboxymethyl cellulose (NaCMC), and polyvinylpyrrolidone (PVP), or combinations thereof. Other mucoadhesive water-erodable polymers may also be used. The term “polyacrylic acid” includes both uncross-linked and partially cross-linked forms, e.g., polycarbophil.

Similar film-forming water-erodable polymers can also be used. The film-forming water-erodable polymers can optionally be cross-linked and/or plasticized in order to alter its dissolution kinetics.

In some embodiments, the properties of the mucoadhesive polymeric diffusion environment are influenced by its pH. In some embodiments, e.g., when mucoadhesive polymeric diffusion environment comprises buprenorphine, its pH is between about 4.0 and about 7.5. In one embodiment, the pH is between 4.0 and 6.0, more specifically, between 4.5 and 5.5, and even more specifically, between 4.75 and 5.25. In a specific embodiment, the pH is 4.75. It is to be understood that all values and ranges between these values and ranges are meant to be encompassed.

The pH of the mucoadhesive polymeric diffusion environment can be adjusted and/or maintained by methods including, but not limited to, the use of buffering agents, or by adjusting the composition of the device.

Buffering agents suitable for use include, for example, phosphates, such as sodium phosphate; phosphates monobasic, such as sodium dihydrogen phosphate and potassium dihydrogen phosphate; phosphates dibasic, such as disodium hydrogen phosphate and dipotassium hydrogen phosphate; phosphates tribasic, such as trisodium phosphate; citrates, such as sodium citrate (anhydrous or dehydrate) and triethyl citrate; bicarbonates, such as sodium bicarbonate and potassium bicarbonate; acetates, such as sodium acetate, may be used. In one embodiment, a single buffering agent, e.g., a dibasic buffering agent is used. In another embodiment, a combination of buffering agents is employed, e.g., a combination of a tri-basic buffering agent and a monobasic buffering agent. In some embodiments, the amount of buffering agent is present in a composition used to make the mucoadhesive layer is about 1 to 20% of the amount of the agonist drug, e.g., buprenorphine.

As discussed herein, the patch device (e.g., the patch device 20) can further comprise at least one additional non-adhesive polymeric environment, e.g., a backing layer (e.g., the backing layer 24). This layer is disposed adjacent to the mucoadhesive polymeric diffusion environment and functions to facilitate the delivery of the opioid agonist, such as buprenorphine, to the mucosa. This additional layer may comprise the same or a different combination of polymers as the mucoadhesive polymeric diffusion environment or the non-adhesive polymeric diffusion environment.

In some embodiments, the backing layer includes an additional medicament, such as an opioid antagonist, to render the device abuse-resistant. In some embodiments, the opioid antagonist is naloxone. The dose of naloxone that can be incorporated into the device depends, in part, on the desired treatment dosage to be administered and can range from about 100 μg to 5 mg of naloxone for the treatment of dependence, and from 60 μg to 1.5 mg naloxone for the pain indication. In some embodiments, the dose of naloxone is between about 0.0125 mg to about 2.0 mg of naloxone, e.g., 0.0125 mg, 0.0130 mg, 0.0135 mg, 0.0140 mg, 0.0145 mg, 0.0150 mg, 0.0155 mg, 0.0160 mg, 0.0165 mg, 0.0170 mg, 0.0175 mg, 0.0180 mg, 0.0185 mg, 0.0190 mg, 0.0195 mg, 0.02 mg, 0.03 mg, 0.04 mg, 0.05 mg, 0.06 mg, 0.07 mg, 0.08 mg, 0.09 mg, 0.1 mg, 0.145 mg, 0.2 mg, 0.29 mg, 0.3 mg, 0.4 mg, 0.5 mg, 0.58 mg, 0.6 mg, 0.7 mg, 0.8 mg, 0.87 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg or 2.0 mg of naloxone. In one embodiment, the dose is 0.145 mg of naloxone. In another embodiment, the dose is 290 μg of naloxone. In another embodiment, the dose is 580 μg of naloxone. In yet another embodiment, the dose is 870 μg of naloxone. In some embodiments, the amount of buprenorphine and the amount of naloxone disposed in the device are present in a ratio chosen such that the effect of buprenorphine is negated by naloxone if the mixture is injected or snorted. In such embodiment, buprenorphine and naloxone disposed in the device are present in a w/w ratio that ranges between 1:4 and 1:10. In a preferred embodiment, the w/w ratio of buprenorphine to naloxone is 1:4 to 1:6, wherein 1:6 is the most preferred embodiment.

In some embodiments, the backing layer (i.e., the non-adhesive polymeric embodiment; e.g., the backing layer 24) functions as a barrier to facilitate a unidirectional flux of the medicament, e.g., buprenorphine, disposed in the mucoadhesive layer. Upon application to a mucosal surface, a diffusional gradient of a medicament is created towards the mucosa. In another embodiment, the backing layer can serve an erodible polymer that facilitate absorption of buprenorphine in the orophyrangeal tissue. In some embodiments, the backing layer prevents diffusion away from the mucosal surface. In such instances, a majority of the medicament, i.e., at least 50%, flows towards the mucosa. In other embodiments, the non-adhesive polymeric environment may circumscribe the boundaries of the mucoadhesive polymeric diffusion environment thereby ensuring that medicament flows toward the mucosa.

The backing layer (e.g., a water-erodable non-adhesive backing layer) can further include at least one water erodable, film-forming polymer. This layer may optionally include a drug. The polymer or polymers can include polyethers and polyalcohols as well as hydrogen bonding cellulosic polymers having either hydroxyalkyl group substitution or hydroxyalkyl group and alkyl group substitution preferably with a moderate to high ratio of hydroxyalkyl to alkyl group. Examples include, but are not limited to, hydroxyethyl cellulose (HEC), hydroxypropyl cellulose (HPC), hydroxypropylmethyl cellulose (HPMC), hydroxyethylmethyl cellulose (HEMC), polyvinyl alcohol (PVA), polyethylene glycol (PEG), polyethylene oxide (PEO), ethylene oxide-propylene oxide co polymers, ethylene oxide-propylene oxide co-polymers, and combinations thereof. The water-erodable non-adhesive backing layer component can optionally be cross-linked.

In certain embodiments, the non-adhesive backing layer is free of cross-linked polymers. In some embodiments, the non-adhesive backing layer is free of polyacrylic acid. While not wishing to be bound by any specific theory, it is estimated that the residence time is reduced by the absence of said polyacrylic acid. For example, in certain embodiments, the residence time is between 15 and 30 minutes. In a preferred embodiment, the water erodable non-adhesive backing layer includes hydroxyethyl cellulose and hydroxypropyl cellulose.

The patch devices described herein can include ingredients that are employed to, at least in part, provide a desired residence time. In some embodiments, this is a result of the selection of the appropriate backing layer formulation, providing a slower rate of erosion of the backing layer. Thus, the non-adhesive backing layer is further modified to render controlled erodability which can be accomplished by coating the backing layer film with a more hydrophobic polymer selected from a group of FDA approved Eudragit™ polymers, ethyl cellulose, cellulose acetate phthalate, and hydroxyl propyl methyl cellulose phthalate, that are approved for use in other pharmaceutical dosage forms. Other hydrophobic polymers may be used, alone or in combination with other hydrophobic or hydrophilic polymers, provided that the layer derived from these polymers or combination of polymers erodes in a moist environment. Dissolution characteristics may be adjusted to modify the residence time and the release profile of a drug when included in the backing layer.

In some embodiments, any of the layers in the patch devices (e.g., active layer patches 22, 24) may also contain a plasticizing agent, such as propylene glycol, polyethylene glycol, or glycerin in a small amount, 0 to 15% by weight, in order to improve the “flexibility” of this layer in the mouth and to adjust the erosion rate of the device. In addition, humectants such as hyaluronic acid, glycolic acid, and other alpha hydroxyl acids can also be added to improve the “softness” and “feel” of the device. Finally, colors and opacifiers may be added to help distinguish the resulting non-adhesive backing layer from the mucoadhesive polymeric diffusion environment. Some opacifers include titanium dioxide, zinc oxide, zirconium silicate, etc.

The patch device can also optionally include a pharmaceutically acceptable dissolution-rate-modifying agent, a pharmaceutically acceptable disintegration aid (e.g., polyethylene glycol, dextran, polycarbophil, carboxymethyl cellulose, or poloxamers), a pharmaceutically acceptable plasticizer, a pharmaceutically acceptable coloring agent (e.g., FD&C Blue #1), a pharmaceutically acceptable opacifier (e.g., titanium dioxide), pharmaceutically acceptable anti-oxidant (e.g., tocopherol acetate), a pharmaceutically acceptable system forming enhancer (e.g., polyvinyl alcohol or polyvinyl pyrrolidone), a pharmaceutically acceptable preservative, flavorants (e.g., saccharin and peppermint), neutralizing agents (e.g., sodium hydroxide), buffering agents (e.g., monobasic, or tribasic sodium phosphate), or combinations thereof. Preferably, these components are individually present at no more than about 1% of the final weight of the device, but the amount may vary depending on the other components of the device.

In some embodiments, the non-adhesive polymeric diffusion environment, e.g., the backing layer 24, is a buffered environment. In some embodiments the pH of the backing layer is between 4.0 and 6.0, more specifically, between 4.2 and 4.7, and even more specifically, between 4.2 and 4.4. In one embodiment, the pH of the backing layer is 4.25. It is to be understood that all values and ranges between these values and ranges are meant to be encompassed.

The pH of the backing layer can be adjusted and/or maintained by methods including, but not limited to, the use of buffering agents, or by adjusting the composition of the device. In some embodiments, the properties of the polymeric diffusion environment are influenced by its buffering capacity. In some embodiments, buffering agents are included in the mucoadhesive polymeric diffusion environment. Buffering agents suitable for use include, for example, phosphates, such as sodium phosphate; phosphates monobasic, such as sodium dihydrogen phosphate and potassium dihydrogen phosphate; phosphates dibasic, such as disodium hydrogen phosphate and dipotassium hydrogen phosphate; phosphates tribasic, such as trisodium phosphate; citrates, such as sodium citrate (anhydrous or dehydrate) and triethyl citrate; bicarbonates, such as sodium bicarbonate and potassium bicarbonate; acetates, such as sodium acetate, may be used. In one embodiment, a single buffering agent, e.g., a dibasic buffering agent is used. In another embodiment, a combination of buffering agents is employed, e.g., a combination of a tri-basic buffering agent and a monobasic buffering agent. In some embodiments, the backing layer comprises the opioid antagonist. In another embodiment, the backing layer comprises an opioid antagonist that is present as a microencapsulated particle with polymers. These polymers include, but are not limited to alginates, polyethylene oxide, poly ethylene glycols, polylactide, polyglycolide, lactide-glycolide copolymers, poly-epsilon-caprolactone, polyorthoesters, polyanhydrides and derivatives, methyl cellulose, ethyl cellulose, hydroxypropyl cellulose, hydroxyethyl cellulose, hydroxyethylmethyl cellulose, hydroxypropylmethyl cellulose, polyacrylic acid, and sodium carboxymethyl cellulose, poly vinyl acetate, poly vinyl alcohols, polyethylene glycol, polyethylene oxide, ethylene oxide-propylene oxide co-polymers, collagen and derivatives, gelatin, albumin, polyaminoacids and derivatives, polyphosphazenes, polysaccharides and derivatives, chitin, chitosan bioadhesive polymers, polyacrylic acid, polyvinyl pyrrolidone, sodium carboxymethyl cellulose and combinations thereof.

Many alterations and modifications may be made by those having ordinary skill in the art, given the benefit of present disclosure, without departing from the spirit and scope of the methods and devices described herein. Therefore, it must be understood that the illustrated embodiments have been set forth only for the purposes of example, and that it should not be taken as limiting the invention defined by the following claims. The following claims, therefore, are to be read to include not only the combination of elements which are literally set forth but also all equivalent elements for performing substantially the same function in substantially the same way to obtain substantially the same result.

1. A pharmaceutical agent delivery patch comprising one or more corner edge segments, an effective amount of an opioid drug for the management of pain and/or opioid dependence,
wherein the opioid drug is delivered to a mucosal surface in a unidirectional manner,
wherein the corner edge segments comprises one or more of a rounded corner edge segment, a circular corner edge segment or a diamond corner edge segment, and
wherein when dissolution testing is preformed comprising
positioning the pharmaceutical agent delivery patch in a dissolution basket comprising a cavity such that the patch is supported by a patch device holder and at least a portion of the patch is spaced apart from a sidewall and a bottom wall;
immersing the dissolution basket and the patch therein in a dissolution fluid; and
moving the dissolution basket with the patch therein in the dissolution fluid to generate a flow of the dissolution fluid across the patch device.

2.-87. (canceled)
